SEQUENCE LISTING (291 sequences)

SEQ ID NO: 1 - DNA, 201 bp, Zea mays
gagaaacaggtcatccacagggacttcaaggcgtccaacatcctcctcgactcggtaatcttcagcaaaagcaagcatgcgatatgaaggtcttctccttrgctaccatgtccactaaccaaagacatgccaggatttcaccgcgaagctgtcggacttcgggctcgccaagaacgggccctccgcggggacgtcgcacgt

SEQ ID NO: 2 - DNA, 201 bp, Zea mays
cagctcctgttaacacctgtcacctactgcaagttgcgagccacacgcgggggaggctaaggtcgggctcgagttttatagaggaggaacacgacaagggrtgtaaggtccccaggggacaaggccggagagaaacgcgcgcgatgtagaggagtgcaagtgacctgtgaacgcccattcccggacacaacaatacttgtt

SEQ ID NO: 3 - DNA, 201 bp, Zea mays
cggcttgtgcggcgtacgtgtttgtgggactctccatccatccccagcacgtcactctctgcttggacgaacgcacgcagatgcagacgcagtggaccatkcgtagtaccgctaggttgtgcgtgttctctagtgacgatacgactgacgacacgaacagatcagccaggccagccaagaagcgcagccacaggcgatccg

SEQ ID NO: 4 - DNA, 201 bp, Zea mays
acgacgagggcgtgcttgtggtagaagcagccgtctcgggcgacctggccgacgtgctcggcgacggtatggccaccgccaacgtcgacgagctgtacccsacgcctccagaggtaactacacgtacgtcgcatgcacgtacgtacgcatgtgtatacctacactgtcatgttcctgacaccaacactttacaggacaact

SEQ ID NO: 5 - DNA, 201 bp, Zea mays
gaacctatgatgcatcacctaataaagcattgggtgactccacaaaccaaacacaccactagagtttatgatctttgtggcttgactagtgagcattcttrtttcctcacgagatagttgcaatggacaagatgccatggttgacacgccagcaagaaggcctaaacaagcatgatgctgcagtggcatatgttggctgat

SEQ ID NO: 6 - DNA, 201 bp, Zea mays
gtgaatgcattcttacatggacgccggccggtaaggtgtgtgtgcaaggttctgtacacagtacactgggacagggtaaagagctgataaagatgccaagwatctctgtgcgtacctttcttcaccttgaagctccagctagtagtaacatgcattgccaaagcaactgtctgaagcagtggagaactggccggaggctga

SEQ ID NO: 7 - DNA, 121 bp, Zea mays
ttatggcactcgtctactacatatctatctatggtcgtgagcagaaagatgggtagtacarctcatgcgcgtttccagcaaacaatgacaggcagcgcgaaaatccaaacacttggtggtt

SEQ ID NO: 8 - DNA, 201 bp, Zea mays
acactcaccatggcgcggcgctcgggagcgaaccctgggaagccaccggcctcgatgtcggtcctgctccctcggaacacaaagctcatgatgatcacaaracgctgccaccgacactttaaggggcaagattggctccaaactccaaagccccacgaagcaacgaattggccagtgaacctgaggcacagacaaatataa

SEQ ID NO: 9 - DNA, 201 bp, Zea mays
tctaccaaggtagctacttgttaacggctcgataatgctgcttgtaccataaactttgtgtgatgcaaatttcagtcagtgccattctgaccaatgtattrcgatggtagaccgaccttccttctgcagggtggaaccaaagatttttgatgaattatttggcgtgccacgcagcagatttgaccgggaagcttgcaggtc

SEQ ID NO: 10 - DNA, 121 bp, Zea mays
agagcgggtgctgatgtgatgcggatccctacgaccttgtcattactagggcacaccacgmgatcgacgtggtctccaattctcccaggaccacatgtcagagacccaccggcctgcaccg

SEQ ID NO: 11 - DNA, 174 bp, Zea mays
aatgctgcaggcgtcctttagtccgtatcacggtcgtccggagagagcgatggatcaaacattgcgattccagwtccatgctagaatagtcttggattcgagtacaaattacgtaattttagctctaatctacatcgacacatatagattatataaagtgaatactactacata

SEQ ID NO: 12 - DNA, 201 bp, Zea mays
aagccacgccagatggccttggccctggccccgctccacgtcctcgcccgcagccaccactgcagagcaaagacacgaaccggtctcccatacgtgagtcwctactctagtgcaacggcaagaactcatgaggtgaaagatgcggccacttacgcgagtggtgtgcgtacggtaattccgcgtagtacgtgcagtccctgt

SEQ ID NO: 13 - DNA, 201 bp, Zea mays
aggggcacgcccggggggccaacgacgaccaccaacgccaatgcaacctgcctgtgcatgccttgacagcaggggaaggaagaagtaactgcatcgtttaktttgctgcatttccaagccaatatattaatctcactgagatcgatcggtcgcctgctgcggacggatccttcgcccggccggggctcgtggtcgccgtcc

SEQ ID NO: 14 - DNA, 201 bp, Zea mays
ggtctgcgctgcgggtggtggacgcgttcctcgtgccgcggttccactacgatccaatcaagaaagtattttacgagtatgtgaacgctgactcttctttrtggatctgggaaattctccacggggacttgagtattggttcgttctttttaggcttctcaattgaaccggggttagagattgctattttgccaaaattgg

SEQ ID NO: 15 - DNA, 201 bp, Zea mays
atgcatcaagaagaaatcagcaaaacggacatgatcattgcaaacctttttcataggtgcacaagttacataattgacagacaacaacagcatcgaatttrtaagccaaattctgcaaaaagatcggaattaaaacaagctgaacagagcgtgtgcaaccatgggctggctggctggctggttcctctcctcaccgtcgtc

SEQ ID NO: 16 - DNA, 121 bp, Zea mays
gtgtgcgtttacaatccaggatcaggagtgtcgcgaacgacttcatccacggatgtcctaygaacgacacgactcgccgcatcgtcatgacgctccagcacctccatcaggccttcgtccg

SEQ ID NO: 17 - DNA, 201 bp, Zea mays
  misc_feature (118)..(118): n is a, c, g, or t
actgtcattagcattcgtcttcttcaaccccggcgaaacactctgtcagtctgtccattgcactgttataaccactctccagcatgcaaccaagcatagcmaactcgactatgcaacnggtgagtcatatttcntctttgttcgtgagtccaatcttatcggccccaaatcaatttcttcctcctgactgaaactgattat

SEQ ID NO: 18 - DNA, 201 bp, Zea mays
agaagaagaagacgaagaaaaaaaacgaccaacattcaaatcaaccaacggcaatgcaagttgctaggaataaatgcgtgcgtggacagcttggagcatgyattgtagtgtagatcgtgtccaaataaatccgggcacaaggaggttttggccactgcattattgttctgaatcaacaataacggccgaagagtgagacga

SEQ ID NO: 19 - DNA, 121 bp, Zea mays
tcgctgcatcgctaagctaggaaaagatccggatctcgagagacacacatcagtagaactkgacggctcgaagatcaccaacctgtttttattttctagcattaaaaaaagctttaccggc

SEQ ID NO: 20 - DNA, 201 bp, Zea mays
cttgctatggattgaaactcagccaatccacatatggattctgatgaaaccgaaagcccttactgaatcattcgagcgctcttgaaacagtagtgctacarggtatccagtgagcgttacttaaacaagcatcctccccccaaaatcacaagatacacatttgcagctgcaaactatacaaaatgttgattctggattccg

SEQ ID NO: 21 - DNA, 201 bp, Zea mays
cggtaaggtgtgtgtgcaaggttctgtacacagtacactgggacagggtaaagagctgataaagatgccaagaatctctgtgcgtacctttcttcaccttsaagctccagctagtagtaacatgcattgccaaagcaactgtctgaagcagtggagaactggccggaggctgagctctcgatcggcgttgctcgtgcagtg

SEQ ID NO: 22 - DNA, 201 bp, Zea mays
gggcccgggggcaggttgtggcggctgcgggcgaagaagaagtggaggtacagggaggagacgatgaggatggaagatatcagcgtgtagaggtagaagarctccatggtgagctactgagctgagctctgtggcacgtgcgcattactatgcgacgtggtctcgttcttgtagaggcggacgccggacaagctccacggc

SEQ ID NO: 23 - DNA, 201 bp, Zea mays
ctgcctccagaaccttttctggaaaccactggagcccggctgttgcgccgtgggccgtggcggcgtgcgacgacggagtgacggacagtgtcaccgtgatsatgagatagatcatggcctattcgatttaaagttgcaggacagcgtggtgtcgcccttggccggacccaggtctacatgtccgtgaagcccttgagttga

SEQ ID NO: 24 - DNA, 201 bp, Zea mays
ttggtgtgctgcgcccgccacgctccaaccgccttgtgtttctgtttctgaattctgagatcacagtgagagaggcgacaacatctcgcatggatggtctmgtattgggtgattcactgatacagcgtgaaacaacaaaaaaggcatgctccaaacaagcctgggggcactgaccccgcagtgccggatctagcagattgt

SEQ ID NO: 25 - DNA, 201 bp, Zea mays
atgtgctcttcttctacgtgttttcttgcggctgggctcagcagttgcccgtgcagccgtgctcgtctagaccgtgtccatgctgctgcaagcaccaacakggttttactagtattttccacggcatctttcgcagagctcgtagctgcatgtctgagtgcctgcgatggcgattgaagaataccgtatgggcatatatat

SEQ ID NO: 26 - DNA, 201 bp, Zea mays
cggctcaactacacgggccgctcgtctcagctttcctcccgccgtagctcacctactaaacaagtgtaaaatcacgtaagatcaccgcgcgtgtcctaggmcgtacaggttggtactctaaactcgctccatgtgtgcccgccatgtgcggagacggtggtgatctaggctatagattcagccagttgcgtccggtcatct

SEQ ID NO: 27 - DNA, 201 bp, Zea mays
tggcctccgtggcgccggttctggaggcagggctggggctaggcggtggcggtggcggcgatctggccacgtcctccagcgccacaatcgtgagtgcttgygcttcggttccaaaatctctttcctgaatcatgatcgcccctggacaagtctaataatttcaagatgatgtgcatgcggtttgtgttcgtatgtgcacgc

SEQ ID NO: 28 - DNA, 201 bp, Zea mays
tggtcactaacctccatcatcatcacgccataactcatgggttgctgctgatagttacaggaaagtgcagggcaaaagctgggtgaagttgaagaaatgcmaaaagtccagacaccaaaatacgtattgctgcggagcaaatggaatcgtagtggacaaacggaattgctggccggcagctagtctgccactcgatccatc

SEQ ID NO: 29 - DNA, 201 bp, Zea mays
caacaggaaagataataattaattagcaagaggtacccccaagccttcttgcttcgttcagtagacagtctacaactgaagctgaattgtcagtgacaggraagactctgccatcgtcctccgtctgacagtgacatgacaaccaacatgaggaagggagcaacattagcaaccagtagctgcggaggaatgcatacagaa

SEQ ID NO: 30 - DNA, 201 bp, Zea mays
ggacttgggagcgaccatcgccggacccggattcgtcgctggtgctcacgtgcgtcaacgtcgctacgtgatatctgacctaacggagacgtcagagtggygcagaaataaaggagaaagaatccgtagagtagcaagttcggcaggggttggcgtaccccaactcaattcaagtgctcgacgaggcaccggaattctacg

SEQ ID NO: 31 - DNA, 121 bp, Zea mays
ccgtgtgcggagaggaatgctcgtgctgtcaccgcgagaaactgctccgcggctgctcgascagtggttgttggtttcagtgattgatttgcttcgttgcaggcaggcccgtccatggagg

SEQ ID NO: 32 - DNA, 201 bp, Zea mays
aatcgcgcacctgccggtgttcgccgcgtggaccgacgcatgaacaaacatccgtgtcattaccagggtacggttcttttggtttgcttagagcgtgcttwttagctgggtaaggttagctgcgtagcggtggtctgatcagcatatgtgagaggaactagccatgcgtgtctgctttggcctcctgcgattcctcctcca

SEQ ID NO: 33 - DNA, 201 bp, Zea mays
ggtgcttgaaccgtgaatatgtgaggctcatgttgggtttcaactctagccaactaaaacttgcttgggagtgaaggctttgttgttgctgttgtattggycacaccagttccaaaaggtggttttggggccatcggtactctcaacgtatcaaatgatttcttgttgtctttcctttgcagtccctgctgtgtgacccaa

SEQ ID NO: 34 - DNA, 121 bp, Zea mays
caaattgctaagctgcctgcccgggttcggtttatcctgttggaaaacgactacagagctyctcctgtttctcgtgtcatttttagtccactgtttctcggtttcaagtacaccaactaat

SEQ ID NO: 35 - DNA, 121 bp, Zea mays
gcgtgggaagcaacaaatgacggcgaacgatgggcgaatacaaaaccttactggaacatgrcgtcgcggatatgcatctccgtgtgggttccactgcgaaaacgacggacgtattcacatt

SEQ ID NO: 36 - DNA, 201 bp, Zea mays
gatgcagtgtgtttgtgtgtgtgtgtgtgtgtgtgtcatgccatcacgaagcctgaatctgaataactcttgttgcttctccattgcatcaggttagcccygacctctacggggataacaacacgaggctcttcacgtactggacagtaagcggctaagcgccttaattattatatattctatctatcttaccgccattgc

SEQ ID NO: 37 - DNA, 201 bp, Zea mays
acacatgatagcatgaaccatgtggttgtcgattgatgccgacgttgcagtcgctcgggcgggctggcatgggccgcatggctgttctttgcttcttcttscacgcacgcatgcaaacatgccgacgtcttaccgaatccagttgaacaaattcagctagcagcggtcggtatcaagagtagtagctgtaacaataactag

SEQ ID NO: 38 - DNA, 201 bp, Zea mays
tagtgcactacggaaacgcgagagaagaggcagacggcggcggcggccatggatttccactccctctcccggcgagaacttcaggcgctctgcaagagcarcggcatccgtgccaacatgtccaacgcggccatggcggaagcgcttaggtgcctccccgcggtgagcctatcctcgatcgatccccttcctttcctcctg

SEQ ID NO: 39 - DNA, 121 bp, Zea mays
gatcaggtatttgcacaagatgtgacatttgagccccggcccacacaatccacacatgcascatgaccaacttcgctgtcgtccatggaccatggggtcgcattttcgggcttcccagcgt

SEQ ID NO: 40 - DNA, 201 bp, Zea mays
atgcgtttgttaacggttggaaagaagaggtctcgggggacacaactccttgttgaagaatacaaggagaagctcggtcactattgcgagttcgaggacastcttatcaggtccaacccaaagcttacaaggtatgatcctccgactttgaagccggtagtcagatacatctataacaggcgaccctcactgcagtgtgca

SEQ ID NO: 41 - DNA, 201 bp, Zea mays
cctaggtacccacctaccccctaggcgaggcggtaccccgtcgcccagcgcataagcgcgcccaggcgagcacggaatagctccttttcgaatacactcgrgcgaagcttagatctgatctcaacgaacctgcagcttgctgcagccactcctcgccggcgatggatttcatgaaggttttcgatcagactgtgcgtgaaa

SEQ ID NO: 42 - DNA, 121 bp, Zea mays
tatgtatgttcctgaggacaggaaacacaggcagcaccaacgttttggtagtagcgcggasagtactacgtactgcgcaactccgcccgcgatgtgttcgttcgtcaatatctccggctag

SEQ ID NO: 43 - DNA, 201 bp, Zea mays
cttaagtactagtttgatgacaagaaaaattgaatggatctatggacggatcgatgggcgatgtcgatggagagcccagagagccgggcatgtatcttctycatcgccgagttaataacacaacatgagtctcgaccgatcgacccgcaagccgttgtgcacacgcgcgcgcgcgtgccgaccgatccagcccaactagac

SEQ ID NO: 44 - DNA, 121 bp, Zea mays
accaaaccaagcgcaggcggcaggcggtttgcttacctggcaggctcccttctcctttcckcacccagctccatcccgtgatcctgatccatcgatccatggcggcggccaataagtccat

SEQ ID NO: 45 - DNA, 201 bp, Zea mays
caggctctgcaaggtgaacggcgacgaggccgagttcacgtacaaggacggcgtggtcaccgtcgacgtgccgtggtcggggtcgtcgtcgaagctgtgtygcgtccagtacgtctactgagccggacgggccgatgactctgctccctgctggcctgctcaggacgcaataatctaatgtttagagcttaccaagtttta

SEQ ID NO: 46 - DNA, 121 bp, Zea mays
atacaaagacacaacaagatatggaactaggagaaacaacacccggagagaaccaggttcstggagagttgctgctcctcatggagagctggtatccaattcagaggttctaatacactgc

SEQ ID NO: 47 - DNA, 121 bp, Zea mays
ccaatccatttcttcccactccttctcggtcggcgacggaagggagacgaagcaagccgtsaccgtgagaaaaccaggtaaagccacaccacaatggccatgccatttttttctactagtt

SEQ ID NO: 48 - DNA, 201 bp, Zea mays
tcttctttccttttttcctttttttttcttcttttccccggccatggttcctttgctttccagcagttctctgctggatgtgatgtatccattgttgcaakcatggccttgcattggctacctctatacctgctacaaaactactgcaacgcctatatatacttggggtgaggaacatgtgaatgcaagctccggctatca

SEQ ID NO: 49 - DNA, 201 bp, Zea mays
cctgcgagttggtcgtgttggccgcgtcgctcccggccgacggccacccgctctccgacacgacgacggtgacgttcccggccccggcgttctccagcgcsgagtagaagcagtcgaccatcgcatcgaacaggctgtcgtagctgtacccgccgtcttgcaccaccaccgtgccggcgggcatggcgaggagcgcgtagt

SEQ ID NO: 50 - DNA, 201 bp, Zea mays
ctgttgtttctatacacgtagatttataaatgtaagtatttgcttccgggtttcgagtctgctatgcgcgctgtacagcatctacatgcgtacgcacccgmagagagtccagaccatctagtctacacggaatgcgccttgtcctcgacgacgtggtcgtggtcgtggatggccccggacacctcctgcacggtgtcgcag

SEQ ID NO: 51 - DNA, 201 bp, Zea mays
ttaaatagatgagttgagagtcacaaacctgcaagaacttgcgcttctgagaccagtgcctagggacaggtactgtatttcggtacgccttgagatatacragcaattttggatctgcagcagttgcatcccagacctggaattggaaagaaaatgagatgaagatagatggtctatacatagttggtttttatcaacaaa

SEQ ID NO: 52 - DNA, 201 bp, Zea mays
caggaaaacgctagggtctgtcggtcgggacttgggacatatacgagtgtaacatgaagagtagagtgaggctgtgtccactgtccagtagatagtacatrtgaccgtgcaaattgcacagtatatatcactgtctgtaatgtaatgtgaagtttgagatgaggcaggagagccggtagagacagcggaacggaagagcgg

SEQ ID NO: 53 - DNA, 201 bp, Zea mays
cattaaaacacactacaatgattcagccacttccactgcatcgagataactttcttgtaaagtactttcagctagctgcgtaactgacgaacaatttacayttgtgctatctttacaaaagctagcgctgtagttcatctgcgtcgttttacaacgagagaagtaacttcctccaacaccaagatgagctgatgtccagat

SEQ ID NO: 54 - DNA, 121 bp, Zea mays
tatgcatgtattatgcatactaacttcgattgcttttctaaatttataggttgtacatgtrtacacccatgtgctttactgggtcggaccctactcatcctccacactaaggccatctcca

SEQ ID NO: 55 - DNA, 121 bp, Zea mays
cgaggccaagagaagactgggtaatcgatcaccacttgctggtcatgcgtctctctgtggkgcgatgcttttgttctttctgtctctttcgcacttactaactcaaacgggttgatttctt

SEQ ID NO: 56 - DNA, 121 bp, Zea mays
gatgcttttgttctttctgtctctttcgcacttactaactcaaacgggttgatttcttggygtcgccatggaaccgtgtcgatcgacagctaaacagcagaagaagaagagccggaagtga

SEQ ID NO: 57 - DNA, 121 bp, Zea mays
ttagggaagccccctgacgctgcatactgtgagggagatgagtacatctggcctggatatrttcctggtcctggggctgggggatagactgagttgccgggattccagttctgcacatgtc

SEQ ID NO: 58 - DNA, 201 bp, Zea mays
gagcgcgtgaccgatccaactgctgaaaagaatcacgatctcgcaacaaacaaacagcctctcccgtgtgttcatccattttctcttcgcttactcgtgastgcatgtgtatgcggtcataatagatggccacgcagctagcaccacccggacgggacggaagcgagcacgctgatattttgatcataacaattatcaaga

SEQ ID NO: 59 - DNA, 201 bp, Zea mays
gtgtgggtgggggcgtggggcagcaggacggtttcccactactgcatcgtagaaggggaattgatgaatgccacggagcacggagatgctgagcgaacaayggaggatagacgtacaatacaagagctggaaagaataattctgatccgcccgttgcagtggtacggcggtgccgtccgttggtggcgaaacgggcgccgg

SEQ ID NO: 60 - DNA, 121 bp, Zea mays
aggttttcaaactagcctaaaaaatagaggcgacctgcagaaagatacggacacgactcgstcggaatacaactgttggaagttgaggcaaaccaaacccaaatagttggagccacacatg

SEQ ID NO: 61 - DNA, 45 bp, Artificial Sequence
  Primer Sequence
gaaggtgaccaagttcatgctctttggttagtggacatggtagct

SEQ ID NO: 62 - DNA, 42 bp, Artificial Sequence
  Primer Sequence
gaaggtcggagtcaacggatttggttagtggacatggtagcc

SEQ ID NO: 63 - DNA, 26 bp, Artificial Sequence
  Primer Sequence
gcatgcgatatgaaggtcttctcctt

SEQ ID NO: 64 - DNA, 45 bp, Artificial Sequence
  Primer Sequence
gaaggtgaccaagttcatgcttttgaggccccttgttatgcatta

SEQ ID NO: 65 - DNA, 44 bp, Artificial Sequence
  Primer Sequence
gaaggtcggagtcaacggattttgaggccccttgttatgcattg

SEQ ID NO: 66 - DNA, 30 bp, Artificial Sequence
  Primer Sequence
gaccaggtacttttctgaaacttacttgtt

SEQ ID NO: 67 - DNA, 42 bp, Artificial Sequence
  Primer Sequence
gaaggtgaccaagttcatgcttgtcccctggggaccttacat

SEQ ID NO: 68 - DNA, 41 bp, Artificial Sequence
  Primer Sequence
gaaggtcggagtcaacggattgtcccctggggaccttacac

SEQ ID NO: 69 - DNA, 25 bp, Artificial Sequence
  Primer Sequence
gggctcgagttttatagaggaggaa

SEQ ID NO: 70 - DNA, 43 bp, Artificial Sequence
  Primer Sequence
gaaggtgaccaagttcatgctgcacaacctagcggtactacga

SEQ ID NO: 71 - DNA, 42 bp, Artificial Sequence
  Primer Sequence
gaaggtcggagtcaacggattcacaacctagcggtactacgc

SEQ ID NO: 72 - DNA, 23 bp, Artificial Sequence
  Primer Sequence
cagatgcagacgcagtggaccat

SEQ ID NO: 73 - DNA, 42 bp, Artificial Sequence
  Primer Sequence
gaaggtgaccaagttcatgctgtagttacctctggaggcgtg

SEQ ID NO: 74 - DNA, 42 bp, Artificial Sequence
  Primer Sequence
gaaggtcggagtcaacggattgtagttacctctggaggcgtc

SEQ ID NO: 75 - DNA, 21 bp, Artificial Sequence
  Primer Sequence
cgccaacgtcgacgagctgta

SEQ ID NO: 76 - DNA, 46 bp, Artificial Sequence
  Primer Sequence
gaaggtgaccaagttcatgctgtggcttgactagtgagcattctta

SEQ ID NO: 77 - DNA, 44 bp, Artificial Sequence
  Primer Sequence
gaaggtcggagtcaacggattggcttgactagtgagcattcttg

SEQ ID NO: 78 - DNA, 26 bp, Artificial Sequence
  Primer Sequence
gtccattgcaactatctcgtgaggaa

SEQ ID NO: 79 - DNA, 47 bp, Artificial Sequence
  Primer Sequence
gaaggtgaccaagttcatgctgtaaagagctgataaagatgccaagt

SEQ ID NO: 80 - DNA, 47 bp, Artificial Sequence
  Primer Sequence
gaaggtcggagtcaacggattgtaaagagctgataaagatgccaaga

SEQ ID NO: 81 - DNA, 25 bp, Artificial Sequence
  Primer Sequence
ggtgaagaaaggtacgcacagagat

SEQ ID NO: 82 - DNA, 44 bp, Artificial Sequence
  Primer Sequence
gaaggtgaccaagttcatgctgtttgctggaaacgcgcatgagt

SEQ ID NO: 83 - DNA, 41 bp, Artificial Sequence
  Primer Sequence
gaaggtcggagtcaacggatttgctggaaacgcgcatgagc

SEQ ID NO: 84 - DNA, 25 bp, Artificial Sequence
  Primer Sequence
gtcgtgagcagaaagatgggtagta

SEQ ID NO: 85 - DNA, 42 bp, Artificial Sequence
  Primer Sequence
gaaggtgaccaagttcatgcttaaagtgtcggtggcagcgtt

SEQ ID NO: 86 - DNA, 41 bp, Artificial Sequence
  Primer Sequence
gaaggtcggagtcaacggattaaagtgtcggtggcagcgtc

SEQ ID NO: 87 - DNA, 26 bp, Artificial Sequence
  Primer Sequence
cctcggaacacaaagctcatgatgat

SEQ ID NO: 88 - DNA, 43 bp, Artificial Sequence
  Primer Sequence
gaaggtgaccaagttcatgctaggaaggtcggtctaccatcgt

SEQ ID NO: 89 - DNA, 42 bp, Artificial Sequence
  Primer Sequence
gaaggtcggagtcaacggattggaaggtcggtctaccatcgc

SEQ ID NO: 90 - DNA, 26 bp, Artificial Sequence
  Primer Sequence
cagtcagtgccattctgaccaatgta

SEQ ID NO: 91 - DNA, 44 bp, Artificial Sequence
  Primer Sequence
gaaggtgaccaagttcatgctagaattggagaccacgtcgatct

SEQ ID NO: 92 - DNA, 43 bp, Artificial Sequence
  Primer Sequence
gaaggtcggagtcaacggattgaattggagaccacgtcgatcg

SEQ ID NO: 93 - DNA, 26 bp, Artificial Sequence
  Primer Sequence
ggatccctacgaccttgtcattacta

SEQ ID NO: 94 - DNA, 47 bp, Artificial Sequence
  Primer Sequence
gaaggtgaccaagttcatgctgatggatcaaacattgcgattccaga

SEQ ID NO: 95 - DNA, 47 bp, Artificial Sequence
  Primer Sequence
gaaggtcggagtcaacggattgatggatcaaacattgcgattccagt

SEQ ID NO: 96 - DNA, 29 bp, Artificial Sequence
  Primer Sequence
tgtactcgaatccaagactattctagcat

SEQ ID NO: 97 - DNA, 42 bp, Artificial Sequence
  Primer Sequence
gaaggtgaccaagttcatgctcggtctcccatacgtgagtca

SEQ ID NO: 98 - DNA, 42 bp, Artificial Sequence
  Primer Sequence
gaaggtcggagtcaacggattcggtctcccatacgtgagtct

SEQ ID NO: 99 - DNA, 25 bp, Artificial Sequence
  Primer Sequence
gagttcttgccgttgcactagagta

SEQ ID NO: 100 - DNA, 50 bp, Artificial Sequence
  Primer Sequence
gaaggtgaccaagttcatgcttaatatattggcttggaaatgcagcaaaa

SEQ ID NO: 101 - DNA, 48 bp, Artificial Sequence
  Primer Sequence
gaaggtcggagtcaacggattatatattggcttggaaatgcagcaaac

SEQ ID NO: 102 - DNA, 26 bp, Artificial Sequence
  Primer Sequence
gggaaggaagaagtaactgcatcgtt

SEQ ID NO: 103 - DNA, 44 bp, Artificial Sequence
  Primer Sequence
gaaggtgaccaagttcatgctcgtggagaatttcccagatccat

SEQ ID NO: 104 - DNA, 44 bp, Artificial Sequence
  Primer Sequence
gaaggtcggagtcaacggattcgtggagaatttcccagatccac

SEQ ID NO: 105 - DNA, 26 bp, Artificial Sequence
  Primer Sequence
cgagtatgtgaacgctgactcttctt

SEQ ID NO: 106 - DNA, 47 bp, Artificial Sequence
  Primer Sequence
gaaggtgaccaagttcatgctgacagacaacaacagcatcgaattta

SEQ ID NO: 107 - DNA, 47 bp, Artificial Sequence
  Primer Sequence
gaaggtcggagtcaacggattgacagacaacaacagcatcgaatttg

SEQ ID NO: 108 - DNA, 30 bp, Artificial Sequence
  Primer Sequence
aattccgatctttttgcagaatttggctta

SEQ ID NO: 109 - DNA, 41 bp, Artificial Sequence
  Primer Sequence
gaaggtgaccaagttcatgctgcggcgagtcgtgtcgttca

SEQ ID NO: 110 - DNA, 40 bp, Artificial Sequence
  Primer Sequence
gaaggtcggagtcaacggattcggcgagtcgtgtcgttcg

SEQ ID NO: 111 - DNA, 25 bp, Artificial Sequence
  Primer Sequence
aacgacttcatccacggatgtccta

SEQ ID NO: 112 - DNA, 44 bp, Artificial Sequence
  Primer Sequence
gaaggtgaccaagttcatgctccagcatgcaaccaagcatagca

SEQ ID NO: 113 - DNA, 43 bp, Artificial Sequence
  Primer Sequence
gaaggtcggagtcaacggattcagcatgcaaccaagcatagcc

SEQ ID NO: 114 - DNA, 26 bp, Artificial Sequence
  Primer Sequence
gccgataagattggactcacgaacaa

SEQ ID NO: 115 - DNA, 45 bp, Artificial Sequence
  Primer Sequence
gaaggtgaccaagttcatgctaccaatctggtgcagagctaaatt

SEQ ID NO: 116 - DNA, 45 bp, Artificial Sequence
  Primer Sequence
gaaggtcggagtcaacggattaccaatctggtgcagagctaaatc

SEQ ID NO: 117 - DNA, 22 bp, Artificial Sequence
  Primer Sequence
gcaagcgggagcgtagcttgat

SEQ ID NO: 118 - DNA, 42 bp, Artificial Sequence
  Primer Sequence
gaaggtgaccaagttcatgctcgtggacagcttggagcatgt

SEQ ID NO: 119 - DNA, 42 bp, Artificial Sequence
  Primer Sequence
gaaggtcggagtcaacggattcgtggacagcttggagcatgc

SEQ ID NO: 120 - DNA, 28 bp, Artificial Sequence
  Primer Sequence
cggatttatttggacacgatctacacta

SEQ ID NO: 121 - DNA, 43 bp, Artificial Sequence
  Primer Sequence
gaaggtgaccaagttcatgctgttggtgatcttcgagccgtca

SEQ ID NO: 122 - DNA, 40 bp, Artificial Sequence
  Primer Sequence
gaaggtcggagtcaacggattggtgatcttcgagccgtcc

SEQ ID NO: 123 - DNA, 25 bp, Artificial Sequence
  Primer Sequence
ggatctcgagagacacacatcagta

SEQ ID NO: 124 - DNA, 42 bp, Artificial Sequence
  Primer Sequence
gaaggtgaccaagttcatgctcttgaaacagtagtgctacag

SEQ ID NO: 125 - DNA, 46 bp, Artificial Sequence
  Primer Sequence
gaaggtcggagtcaacggattcgctcttgaaacagtagtgctacaa

SEQ ID NO: 126 - DNA, 29 bp, Artificial Sequence
  Primer Sequence
atgcttgtttaagtaacgctcactggata

SEQ ID NO: 127 - DNA, 45 bp, Artificial Sequence
  Primer Sequence
gaaggtgaccaagttcatgctcatgttactactagctggagcttg

SEQ ID NO: 128 - DNA, 45 bp, Artificial Sequence
  Primer Sequence
gaaggtcggagtcaacggattcatgttactactagctggagcttc

SEQ ID NO: 129 - DNA, 25 bp, Artificial Sequence
  Primer Sequence
ctctgtgcgtacctttcttcacctt

SEQ ID NO: 130 - DNA, 40 bp, Artificial Sequence
  Primer Sequence
gaaggtgaccaagttcatgctcagtagctcaccatggagc

SEQ ID NO: 131 - DNA, 43 bp, Artificial Sequence
  Primer Sequence
gaaggtcggagtcaacggattgctcagtagctcaccatggagt

SEQ ID NO: 132 - DNA, 27 bp, Artificial Sequence
  Primer Sequence
ggaagatatcagcgtgtagaggtagaa

SEQ ID NO: 133 - DNA, 46 bp, Artificial Sequence
  Primer Sequence
gaaggtgaccaagttcatgctcgaataggccatgatctatctcatg

SEQ ID NO: 134 - DNA, 46 bp, Artificial Sequence
  Primer Sequence
gaaggtcggagtcaacggattcgaataggccatgatctatctcatc

SEQ ID NO: 135 - DNA, 23 bp, Artificial Sequence
  Primer Sequence
gtgacggacagtgtcaccgtgat

SEQ ID NO: 136 - DNA, 45 bp, Artificial Sequence
  Primer Sequence
gaaggtgaccaagttcatgctacaacatctcgcatggatggtcta

SEQ ID NO: 137 - DNA, 44 bp, Artificial Sequence
  Primer Sequence
gaaggtcggagtcaacggattcaacatctcgcatggatggtctc

SEQ ID NO: 138 - DNA, 25 bp, Artificial Sequence
  Primer Sequence
cacgctgtatcagtgaatcacccaa

SEQ ID NO: 139 - DNA, 38 bp, Artificial Sequence
  Primer Sequence
gaaggtgaccaagttcatgctgctgcaagcaccaacat

SEQ ID NO: 140 - DNA, 43 bp, Artificial Sequence
  Primer Sequence
gaaggtcggagtcaacggattatgctgctgcaagcaccaacag

SEQ ID NO: 141 - DNA, 29 bp, Artificial Sequence
  Primer Sequence
cgaaagatgccgtggaaaatactagtaaa

SEQ ID NO: 142 - DNA, 46 bp, Artificial Sequence
  Primer Sequence
gaaggtgaccaagttcatgctgagtttagagtaccaacctgtacgt

SEQ ID NO: 143 - DNA, 45 bp, Artificial Sequence
  Primer Sequence
gaaggtcggagtcaacggattagtttagagtaccaacctgtacgg

SEQ ID NO: 144 - DNA, 23 bp, Artificial Sequence
  Primer Sequence
gtaagatcaccgcgcgtgtccta

SEQ ID NO: 145 - DNA, 43 bp, Artificial Sequence
  Primer Sequence
gaaggtgaccaagttcatgctcgccacaatcgtgagtgcttgt

SEQ ID NO: 146 - DNA, 42 bp, Artificial Sequence
  Primer Sequence
gaaggtcggagtcaacggattgccacaatcgtgagtgcttgc

SEQ ID NO: 147 - DNA, 29 bp, Artificial Sequence
  Primer Sequence
tgattcaggaaagagattttggaaccgaa

SEQ ID NO: 148 - DNA, 42 bp, Artificial Sequence
  Primer Sequence
gaaggtgaccaagttcatgctgggtgaagttgaagaaatgcc

SEQ ID NO: 149 - DNA, 46 bp, Artificial Sequence
  Primer Sequence
gaaggtcggagtcaacggattagctgggtgaagttgaagaaatgca

SEQ ID NO: 150 - DNA, 29 bp, Artificial Sequence
  Primer Sequence
gcaatacgtattttggtgtctggactttt

SEQ ID NO: 151 - DNA, 45 bp, Artificial Sequence
  Primer Sequence
gaaggtgaccaagttcatgctgaagctgaattgtcagtgacagga

SEQ ID NO: 152 - DNA, 44 bp, Artificial Sequence
  Primer Sequence
gaaggtcggagtcaacggattaagctgaattgtcagtgacaggg

SEQ ID NO: 153 - DNA, 22 bp, Artificial Sequence
  Primer Sequence
cggaggacgatggcagagtctt

SEQ ID NO: 154 - DNA, 41 bp, Artificial Sequence
  Primer Sequence
gaaggtgaccaagttcatgctaacggagacgtcagagtggc

SEQ ID NO: 155 - DNA, 43 bp, Artificial Sequence
  Primer Sequence
gaaggtcggagtcaacggattctaacggagacgtcagagtggt

SEQ ID NO: 156 - DNA, 29 bp, Artificial Sequence
  Primer Sequence
gctactctacggattctttctcctttatt

SEQ ID NO: 157 - DNA, 46 bp, Artificial Sequence
  Primer Sequence
gaaggtgaccaagttcatgctaatcactgaaaccaacaaccactgg

SEQ ID NO: 158 - DNA, 46 bp, Artificial Sequence
  Primer Sequence
gaaggtcggagtcaacggattaatcactgaaaccaacaaccactgc

SEQ ID NO: 159 - DNA, 22 bp, Artificial Sequence
  Primer Sequence
ctcgtgctgtcaccgcgagaaa

SEQ ID NO: 160 - DNA, 45 bp, Artificial Sequence
  Primer Sequence
gaaggtgaccaagttcatgcttttggtttgcttagagcgtgctta

SEQ ID NO: 161 - DNA, 47 bp, Artificial Sequence
  Primer Sequence
gaaggtcggagtcaacggattcttttggtttgcttagagcgtgcttt

SEQ ID NO: 162 - DNA, 25 bp, Artificial Sequence
  Primer Sequence
tacgcagctaaccttacccagctaa

SEQ ID NO: 163 - DNA, 46 bp, Artificial Sequence
  Primer Sequence
gaaggtgaccaagttcatgctaaaccaccttttggaactggtgtga

SEQ ID NO: 164 - DNA, 43 bp, Artificial Sequence
  Primer Sequence
gaaggtcggagtcaacggattccaccttttggaactggtgtgg

SEQ ID NO: 165 - DNA, 29 bp, Artificial Sequence
  Primer Sequence
agtgaaggctttgttgttgctgttgtatt

SEQ ID NO: 166 - DNA, 44 bp, Artificial Sequence
  Primer Sequence
gaaggtgaccaagttcatgctgttggaaaacgactacagagctt

SEQ ID NO: 167 - DNA, 44 bp, Artificial Sequence
  Primer Sequence
gaaggtcggagtcaacggattgttggaaaacgactacagagctc

SEQ ID NO: 168 - DNA, 29 bp, Artificial Sequence
  Primer Sequence
gaaacagtggactaaaaatgacacgagaa

SEQ ID NO: 169 - DNA, 42 bp, Artificial Sequence
  Primer Sequence
gaaggtgaccaagttcatgctggagatgcatatccgcgacgt

SEQ ID NO: 170 - DNA, 42 bp, Artificial Sequence
  Primer Sequence
gaaggtcggagtcaacggattggagatgcatatccgcgacgc

SEQ ID NO: 171 - DNA, 27 bp, Artificial Sequence
  Primer Sequence
ggcgaatacaaaaccttactggaacat

SEQ ID NO: 172 - DNA, 42 bp, Artificial Sequence
  Primer Sequence
gaaggtgaccaagttcatgctccattgcatcaggttagcccc

SEQ ID NO: 173 - DNA, 44 bp, Artificial Sequence
  Primer Sequence
gaaggtcggagtcaacggattctccattgcatcaggttagccct

SEQ ID NO: 174 - DNA, 25 bp, Artificial Sequence
  Primer Sequence
gtacgtgaagagcctcgtgttgtta

SEQ ID NO: 175 - DNA, 46 bp, Artificial Sequence
  Primer Sequence
gaaggtgaccaagttcatgctcatggctgttctttgcttcttcttc

SEQ ID NO: 176 - DNA, 46 bp, Artificial Sequence
  Primer Sequence
gaaggtcggagtcaacggattcatggctgttctttgcttcttcttg

SEQ ID NO: 177 - DNA, 25 bp, Artificial Sequence
  Primer Sequence
gattcggtaagacgtcggcatgttt

SEQ ID NO: 178 - DNA, 42 bp, Artificial Sequence
  Primer Sequence
gaaggtgaccaagttcatgcttcaggcgctctgcaagagcaa

SEQ ID NO: 179 - DNA, 41 bp, Artificial Sequence
  Primer Sequence
gaaggtcggagtcaacggattcaggcgctctgcaagagcag

SEQ ID NO: 180 - DNA, 23 bp, Artificial Sequence
  Primer Sequence
gcgttggacatgttggcacggat

SEQ ID NO: 181 - DNA, 47 bp, Artificial Sequence
  Primer Sequence
gaaggtgaccaagttcatgctcctctgaacatacatacacagaccat

SEQ ID NO: 182 - DNA, 46 bp, Artificial Sequence
  Primer Sequence
gaaggtcggagtcaacggattctctgaacatacatacacagaccag

SEQ ID NO: 183 - DNA, 27 bp, Artificial Sequence
  Primer Sequence
gytagactgtagctcgcttgtgaatat

SEQ ID NO: 184 - DNA, 43 bp, Artificial Sequence
  Primer Sequence
gaaggtgaccaagttcatgctacgacagcgaagttggtcatgg

SEQ ID NO: 185 - DNA, 43 bp, Artificial Sequence
  Primer Sequence
gaaggtcggagtcaacggattacgacagcgaagttggtcatgc

SEQ ID NO: 186 - DNA, 22 bp, Artificial Sequence
  Primer Sequence
ccggcccacacaatccacacat

SEQ ID NO: 187 - DNA, 44 bp, Artificial Sequence
  Primer Sequence
gaaggtgaccaagttcatgctcactattgcgagttcgaggacac

SEQ ID NO: 188 - DNA, 44 bp, Artificial Sequence
  Primer Sequence
gaaggtcggagtcaacggattcactattgcgagttcgaggacag

SEQ ID NO: 189 - DNA, 26 bp, Artificial Sequence
  Primer Sequence
cttgtaagctttgggttggacctgat

SEQ ID NO: 190 - DNA, 46 bp, Artificial Sequence
  Primer Sequence
gaaggtgaccaagttcatgctgttgagatcagatctaagcttcgct

SEQ ID NO: 191 - DNA, 43 bp, Artificial Sequence
  Primer Sequence
gaaggtcggagtcaacggattgagatcagatctaagcttcgcc

SEQ ID NO: 192 - DNA, 25 bp, Artificial Sequence
  Primer Sequence
gagcacggaatagctccttttcgaa

SEQ ID NO: 193 - DNA, 43 bp, Artificial Sequence
  Primer Sequence
gaaggtgaccaagttcatgctacgttttggtagtagcgcggac

SEQ ID NO: 194 - DNA, 43 bp, Artificial Sequence
  Primer Sequence
gaaggtcggagtcaacggattacgttttggtagtagcgcggag

SEQ ID NO: 195 - DNA, 23 bp, Artificial Sequence
  Primer Sequence
gcggagttgcgcagtacgtagta

SEQ ID NO: 196 - DNA, 45 bp, Artificial Sequence
  Primer Sequence
gaaggtgaccaagttcatgctcacgaacaaaactgcatgaggttc

SEQ ID NO: 197 - DNA, 45 bp, Artificial Sequence
  Primer Sequence
gaaggtcggagtcaacggattcacgaacaaaactgcatgaggttg

SEQ ID NO: 198 - DNA, 29 bp, Artificial Sequence
  Primer Sequence
catggttcttggtcagtttcagaagaaat

SEQ ID NO: 199 - DNA, 44 bp, Artificial Sequence
  Primer Sequence
gaaggtgaccaagttcatgctgagagccgggcatgtatcttctt

SEQ ID NO: 200 - DNA, 43 bp, Artificial Sequence
  Primer Sequence
gaaggtcggagtcaacggattagagccgggcatgtatcttctc

SEQ ID NO: 201 - DNA, 28 bp, Artificial Sequence
  Primer Sequence
actcatgttgtgttattaactcggcgat

SEQ ID NO: 202 - DNA, 41 bp, Artificial Sequence
  Primer Sequence
gaaggtgaccaagttcatgctcacgggatggagctgggtga

SEQ ID NO: 203 - DNA, 40 bp, Artificial Sequence
  Primer Sequence
gaaggtcggagtcaacggattacgggatggagctgggtgc

SEQ ID NO: 204 - DNA, 22 bp, Artificial Sequence
  Primer Sequence
ctggcaggctcccttctccttt

SEQ ID NO: 205 - DNA, 41 bp, Artificial Sequence
  Primer Sequence
gaaggtgaccaagttcatgctcagtagacgtactggacgca

SEQ ID NO: 206 - DNA, 41 bp, Artificial Sequence
  Primer Sequence
gaaggtcggagtcaacggattcagtagacgtactggacgcg

SEQ ID NO: 207 - DNA, 21 bp, Artificial Sequence
  Primer Sequence
gtggtcggggtcgtcgtcgaa

SEQ ID NO: 208 - DNA, 43 bp, Artificial Sequence
  Primer Sequence
gaaggtgaccaagttcatgctatgaggagcagcaactctccag

SEQ ID NO: 209 - DNA, 43 bp, Artificial Sequence
  Primer Sequence
gaaggtcggagtcaacggattatgaggagcagcaactctccac

SEQ ID NO: 210 - DNA, 23 bp, Artificial Sequence
  Primer Sequence
caacacccggagagaaccaggtt

SEQ ID NO: 211 - DNA, 43 bp, Artificial Sequence
  Primer Sequence
gaaggtgaccaagttcatgctccaagcaaaatggtgtccctcc

SEQ ID NO: 212 - DNA, 44 bp, Artificial Sequence
  Primer Sequence
gaaggtcggagtcaacggattaccaagcaaaatggtgtccctca

SEQ ID NO: 213 - DNA, 25 bp, Artificial Sequence
  Primer Sequence
gcatcgggatttcatccgtcatcaa

SEQ ID NO: 214 - DNA, 41 bp, Artificial Sequence
  Primer Sequence
gaaggtgaccaagttcatgctgggagacgaagcaagccgtc

SEQ ID NO: 215 - DNA, 41 bp, Artificial Sequence
  Primer Sequence
gaaggtcggagtcaacggattgggagacgaagcaagccgtg

SEQ ID NO: 216 - DNA, 25 bp, Artificial Sequence
  Primer Sequence
cattgtggtgtggctttacctggtt

SEQ ID NO: 217 - DNA, 47 bp, Artificial Sequence
  Primer Sequence
gaaggtgaccaagttcatgctggatgtgatgtatccattgttgcaat

SEQ ID NO: 218 - DNA, 47 bp, Artificial Sequence
  Primer Sequence
gaaggtcggagtcaacggattggatgtgatgtatccattgttgcaag

SEQ ID NO: 219 - DNA, 25 bp, Artificial Sequence
  Primer Sequence
atagaggtagccaatgcaaggccat

SEQ ID NO: 220 - DNA, 49 bp, Artificial Sequence
  Primer Sequence
gaaggtgaccaagttcatgctgtcatttctatcatcctgaattcaagag

SEQ ID NO: 221 - DNA, 50 bp, Artificial Sequence
  Primer Sequence
gaaggtcggagtcaacggattggtcatttctatcatcctgaattcaagat

SEQ ID NO: 222 - DNA, 25 bp, Artificial Sequence
  Primer Sequence
gacccagccacacaaattggaagat

SEQ ID NO: 223 - DNA, 47 bp, Artificial Sequence
  Primer Sequence
gaaggtgaccaagttcatgctagacatcgtataaaatggtgaagcga

SEQ ID NO: 224 - DNA, 46 bp, Artificial Sequence
  Primer Sequence
gaaggtcggagtcaacggattgacatcgtataaaatggtgaagcgg

SEQ ID NO: 225 - DNA, 25 bp, Artificial Sequence
  Primer Sequence
ggagactgatgctctgttgtgcaat

SEQ ID NO: 226 - DNA, 39 bp, Artificial Sequence
  Primer Sequence
gaaggtgaccaagttcatgctccggcgttctccagcgcc

SEQ ID NO: 227 - DNA, 39 bp, Artificial Sequence
  Primer Sequence
gaaggtcggagtcaacggattccggcgttctccagcgcg

SEQ ID NO: 228 - DNA, 24 bp, Artificial Sequence
  Primer Sequence
cgatgcgatggtcgactgcttcta

SEQ ID NO: 229 - DNA, 44 bp, Artificial Sequence
  Primer Sequence
gaaggtgaccaagttcatgcttcaatggcaagtacacgaggcta

SEQ ID NO: 230 - DNA, 43 bp, Artificial Sequence
  Primer Sequence
gaaggtcggagtcaacggattcaatggcaagtacacgaggctc

SEQ ID NO: 231 - DNA, 25 bp, Artificial Sequence
  Primer Sequence
atcatctgcgcgcttccagtttgaa

SEQ ID NO: 232 - DNA, 45 bp, Artificial Sequence
  Primer Sequence
gaaggtgaccaagttcatgctttgacatccctgtgtaagatgcgt

SEQ ID NO: 233 - DNA, 43 bp, Artificial Sequence
  Primer Sequence
gaaggtcggagtcaacggattgacatccctgtgtaagatgcgg

SEQ ID NO: 234 - DNA, 28 bp, Artificial Sequence
  Primer Sequence
cgtgcacttgcatatcttcatgacaatt

SEQ ID NO: 235 - DNA, 43 bp, Artificial Sequence
  Primer Sequence
gaaggtgaccaagttcatgctatctacatgcgtacgcacccga

SEQ ID NO: 236 - DNA, 41 bp, Artificial Sequence
  Primer Sequence
gaaggtcggagtcaacggattctacatgcgtacgcacccgc

SEQ ID NO: 237 - DNA, 25 bp, Artificial Sequence
  Primer Sequence
aaggcgcattccgtgtagactagat

SEQ ID NO: 238 - DNA, 46 bp, Artificial Sequence
  Primer Sequence
gaaggtgaccaagttcatgctaatgggaacaagtcaaggtatggca

SEQ ID NO: 239 - DNA, 43 bp, Artificial Sequence
  Primer Sequence
gaaggtcggagtcaacggattgggaacaagtcaaggtatggcg

SEQ ID NO: 240 - DNA, 30 bp, Artificial Sequence
  Primer Sequence
ccatcaacagtgttaataagtcatgtagaa

SEQ ID NO: 241 - DNA, 46 bp, Artificial Sequence
  Primer Sequence
gaaggtgaccaagttcatgctatttcggtacgccttgagatataca

SEQ ID NO: 242 - DNA, 43 bp, Artificial Sequence
  Primer Sequence
gaaggtcggagtcaacggatttcggtacgccttgagatatacg

SEQ ID NO: 243 - DNA, 25 bp, Artificial Sequence
  Primer Sequence
ggatgcaactgctgcagatccaaaa

SEQ ID NO: 244 - DNA, 44 bp, Artificial Sequence
  Primer Sequence
gaaggtgaccaagttcatgctgggttatgagtagaaccgtcgca

SEQ ID NO: 245 - DNA, 43 bp, Artificial Sequence
  Primer Sequence
gaaggtcggagtcaacggattggttatgagtagaaccgtcgcg

SEQ ID NO: 246 - DNA, 30 bp, Artificial Sequence
  Primer Sequence
gcgtaaagaaacacggtcataatcatttaa

SEQ ID NO: 247 - DNA, 48 bp, Artificial Sequence
  Primer Sequence
gaaggtgaccaagttcatgctaaaattctccatatgaatctcctccct

SEQ ID NO: 248 - DNA, 46 bp, Artificial Sequence
  Primer Sequence
gaaggtcggagtcaacggattaattctccatatgaatctcctcccg

SEQ ID NO: 249 - DNA, 29 bp, Artificial Sequence
  Primer Sequence
cttttatctaggtgttctgaagctcgaat

SEQ ID NO: 250 - DNA, 47 bp, Artificial Sequence
  Primer Sequence
gaaggtgaccaagttcatgctatatactgtgcaatttgcacggtcat

SEQ ID NO: 251 - DNA, 45 bp, Artificial Sequence
  Primer Sequence
gaaggtcggagtcaacggattatactgtgcaatttgcacggtcac

SEQ ID NO: 252 - DNA, 26 bp, Artificial Sequence
  Primer Sequence
ctgtgtccactgtccagtagatagta

SEQ ID NO: 253 - DNA, 45 bp, Artificial Sequence
  Primer Sequence
gaaggtgaccaagttcatgctacaaacacagtacctcaccctaca

SEQ ID NO: 254 - DNA, 44 bp, Artificial Sequence
  Primer Sequence
gaaggtcggagtcaacggattcaaacacagtacctcaccctacc

SEQ ID NO: 255 - DNA, 25 bp, Artificial Sequence
  Primer Sequence
ttggacgagcctgtgcctttctata

SEQ ID NO: 256 - DNA, 44 bp, Artificial Sequence
  Primer Sequence
gaaggtgaccaagttcatgcttatggtgcaacatcagaggttag

SEQ ID NO: 257 - DNA, 46 bp, Artificial Sequence
  Primer Sequence
gaaggtcggagtcaacggattcttatggtgcaacatcagaggttat

SEQ ID NO: 258 - DNA, 25 bp, Artificial Sequence
  Primer Sequence
gaacggcttctcttaacgcagcaaa

SEQ ID NO: 259 - DNA, 45 bp, Artificial Sequence
  Primer Sequence
gaaggtgaccaagttcatgctgcgtaactgacgaacaatttacat

SEQ ID NO: 260 - DNA, 47 bp, Artificial Sequence
  Primer Sequence
gaaggtcggagtcaacggattctgcgtaactgacgaacaatttacac

SEQ ID NO: 261 - DNA, 28 bp, Artificial Sequence
  Primer Sequence
agcgctagcttttgtaaagatagcacaa

SEQ ID NO: 262 - DNA, 54 bp, Artificial Sequence
  Primer Sequence
gaaggtgaccaagttcatgctattgcttttctaaatttataggttgtacatgta

SEQ ID NO: 263 - DNA, 51 bp, Artificial Sequence
  Primer Sequence
gaaggtcggagtcaacggattgcttttctaaatttataggttgtacatgtg

SEQ ID NO: 264 - DNA, 24 bp, Artificial Sequence
  Primer Sequence
gacccagtaaagcacatgggtgta

SEQ ID NO: 265 - DNA, 45 bp, Artificial Sequence
  Primer Sequence
gaaggtgaccaagttcatgctgccttgagtgttacaagaccaata

SEQ ID NO: 266 - DNA, 45 bp, Artificial Sequence
  Primer Sequence
gaaggtcggagtcaacggattgccttgagtgttacaagaccaatg

SEQ ID NO: 267 - DNA, 22 bp, Artificial Sequence
  Primer Sequence
cagcacacttgagcgggtcctt

SEQ ID NO: 268 - DNA, 46 bp, Artificial Sequence
  Primer Sequence
gaaggtgaccaagttcatgctatgtcgattatctggagaacaggga

SEQ ID NO: 269 - DNA, 44 bp, Artificial Sequence
  Primer Sequence
gaaggtcggagtcaacggattgtcgattatctggagaacagggc

SEQ ID NO: 270 - DNA, 29 bp, Artificial Sequence
  Primer Sequence
gcgtcataatacagtcgcaatcatcattt

SEQ ID NO: 271 - DNA, 47 bp, Artificial Sequence
  Primer Sequence
gaaggtgaccaagttcatgctgagacagaaagaacaaaagcatcgca

SEQ ID NO: 272 - DNA, 46 bp, Artificial Sequence
  Primer Sequence
gaaggtcggagtcaacggattagacagaaagaacaaaagcatcgcc

SEQ ID NO: 273 - DNA, 26 bp, Artificial Sequence
  Primer Sequence
gtaatcgatcaccacttgctggtcat

SEQ ID NO: 274 - DNA, 46 bp, Artificial Sequence
  Primer Sequence
gaaggtgaccaagttcatgctaactcaaacgggttgatttcttggt

SEQ ID NO: 275 - DNA, 46 bp, Artificial Sequence
  Primer Sequence
gaaggtcggagtcaacggattaactcaaacgggttgatttcttggc

SEQ ID NO: 276 - DNA, 25 bp, Artificial Sequence
  Primer Sequence
tgtttagctgtcgatcgacacggtt

SEQ ID NO: 277 - DNA, 43 bp, Artificial Sequence
  Primer Sequence
gaaggtgaccaagttcatgctgtgtgggaacttgtactgcgca

SEQ ID NO: 278 - DNA, 41 bp, Artificial Sequence
  Primer Sequence
gaaggtcggagtcaacggattgtgggaacttgtactgcgcg

SEQ ID NO: 279 - DNA, 24 bp, Artificial Sequence
  Primer Sequence
cttgcagtcgtgcttgtcggagta

SEQ ID NO: 280 - DNA, 46 bp, Artificial Sequence
  Primer Sequence
gaaggtgaccaagttcatgctgatgagtacatctggcctggatata

SEQ ID NO: 281 - DNA, 45 bp, Artificial Sequence
  Primer Sequence
gaaggtcggagtcaacggattatgagtacatctggcctggatatg

SEQ ID NO: 282 - DNA, 20 bp, Artificial Sequence
  Primer Sequence
cccagccccaggaccaggaa

SEQ ID NO: 283 - DNA, 46 bp, Artificial Sequence
  Primer Sequence
gaaggtgaccaagttcatgctcattttctcttcgcttactcgtgac

SEQ ID NO: 284 - DNA, 46 bp, Artificial Sequence
  Primer Sequence
gaaggtcggagtcaacggattcattttctcttcgcttactcgtgag

SEQ ID NO: 285 - DNA, 25 bp, Artificial Sequence
  Primer Sequence
cgtggccatctattatgaccgcata

SEQ ID NO: 286 - DNA, 45 bp, Artificial Sequence
  Primer Sequence
gaaggtgaccaagttcatgctcttgtattgtacgtctatcctcca

SEQ ID NO: 287 - DNA, 45 bp, Artificial Sequence
  Primer Sequence
gaaggtcggagtcaacggattcttgtattgtacgtctatcctccg

SEQ ID NO: 288 - DNA, 23 bp, Artificial Sequence
  Primer Sequence
gcacggagatgctgagcgaacaa

SEQ ID NO: 289 - DNA, 43 bp, Artificial Sequence
  Primer Sequence
gaaggtgaccaagttcatgctgaaagatacggacacgactcgc

SEQ ID NO: 290 - DNA, 43 bp, Artificial Sequence
  Primer Sequence
gaaggtcggagtcaacggattgaaagatacggacacgactcgg

SEQ ID NO: 291 - DNA, 28 bp, Artificial Sequence
  Primer Sequence
gtttgcctcaacttccaacagttgtatt

(19) United States
(12) Patent Application Publication
(10) Pub. No.: US 2015/0159228 A1
(43) Pub. Date: Jun. 11, 2015

(54) MOLECULAR MARKERS ASSOCIATED WITH CULTURE AND TRANSFORMATION IN MAIZE

(71) Applicant: DOW AGROSCIENCES LLC, INDIANAPOLIS, IN (US)

(72) Inventors: Tristan E. Coram, Zionsville, IN (US); Diaa Alabed, Carmel, IN (US); Susan M. Jayne, Zionsville, IN (US); Stephen Foulk, Carmel, IN (US); Rajat Aggarwal, Zionsville, IN (US); Natae Daniels, Brownsburg, IN (US)

(21) Appl. No.: 14/561,478

(22) Filed: Dec. 5, 2014

Related U.S. Application Data
(60) Provisional application No. 61/914,492, filed on Dec. 11, 2013.

(51) Int. Cl. C12Q 1/68; A01H 5/10; A01H 1/04

(52) U.S. Cl. CPC C12Q 1/6895; A01H 1/04; A01H 5/10; C12Q 2600/13; C12Q 2600/156

(57) ABSTRACT

This invention relates to methods for identifying maize plants that having increased culturability and transformability. The methods use molecular markers to identify and to select plants with increased culturability and transformability. Maize plants generated by the methods of the invention are also a feature of the invention.

### CROSS-REFERENCE TO RELATED APPLICATION

This application claims the benefit of U.S. Provisional Application No. 61/914,492 which was filed in the U.S. Patent and Trademark Office on Dec. 11, 2013, the entirety of the disclosure of which is expressly incorporated by reference herein.

### FIELD OF THE INVENTION

The present invention relates to methods useful in selecting for improved culturability and transformability in maize plants.

### BACKGROUND OF THE INVENTION

Maize transformation has historically been practiced using genotypes that are amenable to tissue culture and gene delivery techniques. However, from a product development stand point, creation of transgenic events using these genotypes is undesirable because they are often agronomically poor. In many instances, however, plants with superior agronomic traits, such as elite lines, tend to exhibit poor culturing and transformability characteristics. Thus, there is an opportunity to develop elite inbred lines that can be cultured and transformed at efficiencies suitable for routine use in maize transformation. Advantages of having such a line include faster and more precise event sorting and trait evaluation, faster seed production for trait, yield, and regulatory trials, and faster product development. Elite maize inbred transformation is commonly practiced and gaining this ability is a vital component of improving and industrializing a trait development process.

A methodology for developing elite transformable inbred lines involves introgression of culturability and transformability traits from donor material into the desired elite line. Having genetic markers for these traits would be valuable for aiding this introgression. The culturability and transformability traits in the Hi-II and A188 germplasm have been well studied, and several molecular markers linked to these traits have been identified (Armstrong et al. 1992; Lowe and Chomet 2004; Lowe et al. 2006; Zhao et al. 2008). Hi-II, a novel line with improved culturability and transformability, was developed from an initial cross between A188 and B73 (Armstrong et al. 1991), wherein A188 was the donor of the culturability and transformability traits.

The present invention addresses the need for more culturable and transformable elite lines and provides improved methods to facilitate the development of new, agronomically superior corn lines with enhance culturability and transformability.

### SUMMARY OF THE INVENTION

One embodiment of the present invention is a method for selecting a plant having an altered culturability and transformability characteristic. The method includes the steps of: a) detecting at least one marker nucleic acid; and, b) selecting a plant comprising the marker nucleic acid, thereby selecting a plant having the altered culturability and transformability characteristic. The plant is preferably a maize plant. The altered culturability and transformability characteristic is preferably increased culturability and transformability.

In embodiments of the invention, the marker nucleic acid is selected from the group consisting of DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624. In other embodiments of the invention, the marker nucleic acid is selected from the group consisting of Mo17-13849, DAS-PZ-4414, DAS-PZ-4181, Mo17-100863, Mo17-102037, DAS-PZ-40282, DAS-PZ-915, DAS-PZ-8563, and PZA00991. In further embodiments, the marker nucleic acid is selected from the group consisting of Mo17-13449, DAS-PZ-2605, and DAS-PZ-19040. In other embodiments, the marker nucleic acid is selected from the group consisting of Mo17-10473, PZA00749, DAS-PZ-21197, Mo17-101840, DAS-PZ-16358, Mo17-11830, and PZA03070. In further embodiments, the marker nucleic acid is selected from the group consisting of Mo17-1107, DAS-PZ-11065, magi73697, magi94661, and DAS-PZ-34879. In other embodiments, the marker nucleic acid is selected from the group consisting of PZA00089, PZA02462, DAS-PZ-6897, PZA02676, and KG-2572840. In further embodiments, the marker nucleic acid is selected from the group consisting of PZA01304 and magi95039. In other embodiments, the marker nucleic acid is selected from the group consisting of DAS-PZ-6618 and magi91369. In further embodiments, the marker nucleic acid is selected from the group consisting of PZA01297, DAS-PZ-386, Mo17-1333, PHM448, PZA03697, Mo17-14260, and Mo17-14261. In other embodiments, the marker nucleic acid is selected from the group consisting of, Mo17-12340, DAS-PZ-1425, DAS-PZ-14198, and Mo17-102555.

In embodiments of the invention, at least two marker nucleic acids are selected, preferably, at least three marker nucleic acids are selected, more preferably at least four marker nucleic acids are selected, still more preferably at least five marker nucleic acids are selected, yet more preferably at least six marker nucleic acids are selected, still more preferably at least seven marker nucleic acids are selected, yet more preferably at least eight marker nucleic acids are selected, still more preferably at least nine marker nucleic acids are selected, and yet more preferably at least ten marker nucleic acids are selected.

In yet another embodiment of the invention is a method for selecting a maize plant having increased culturability and transformability, the method comprising: a) detecting at least ten marker nucleic acids, wherein at least one marker nucleic acid is selected from each of ten marker nucleic acid groups (i)-(x):
(i) DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624;
(ii) Mo17-13849, DAS-PZ-4414, DAS-PZ-4181, Mo17-100863, Mo17-102037, DAS-PZ-40282, DAS-PZ-915, DAS-PZ-8563, and PZA00991;
(iii) Mo17-13449, DAS-PZ-2605, and DAS-PZ-19040;
(iv) Mo17-10473, PZA00749, DAS-PZ-21197, Mo17-101840, DAS-PZ-16358, Mo17-11830, and PZA03070;
(v) Mo17-1107, DAS-PZ-11065, magi73697, magi94661, and DAS-PZ-34879 ; (vi) PZA00089, PZA02462, DAS-PZ-6897, PZA02676, and KG-2572840;
(vii) PZA01304 and magi95039;
(viii) DAS-PZ-6618 and magi91369;
(ix) PZA01297, DAS-PZ-386, Mo17-1333, PHM448, PZA03697, Mo17-14260, and Mo17-14261; and,
(x) PZB01899, Mo17-12340, DAS-PZ-1425, DAS-PZ-14198, and Mo17-102555; and, b) selecting a plant comprising the ten marker nucleic acids, thereby selecting a maize plant having increased culturability and transformability. Maize plants obtained by the methods described herein are also contemplated by the present invention.

### BRIEF DESCRIPTION OF FIGURES AND SEQUENCE LISTINGS

The invention can be more fully understood from the following detailed description and the accompanying drawings and Sequence Listing, which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984), which are herein incorporated by reference in their entirety. The symbols and format used for nucleotide and amino acid sequence data comply with the rules set forth in 37 C.F.R. §1.822.

SEQ ID NO: 1 contains the DAS-PZ-12690 SNP and flanking sequence.

SEQ ID NO: 2 contains the DAS-PZ-9388 SNP and flanking sequence.

SEQ ID NO: 3 contains the DAS-PZ-17750 SNP and flanking sequence.

SEQ ID NO: 4 contains the DAS-PZ-20486 SNP and flanking sequence.

SEQ ID NO: 5 contains the DAS-PZ-10624 SNP and flanking sequence.

SEQ ID NO: 6 contains the DAS-PZ-16137 SNP and flanking sequence.

SEQ ID NO: 7 contains the Mo17-10690 SNP and flanking sequence.

SEQ ID NO: 8 contains the DAS-PZ-5621 SNP and flanking sequence.

SEQ ID NO: 9 contains the KG-2507169 SNP and flanking sequence.

SEQ ID NO: 10 contains the Mo17-1018 SNP and flanking sequence.

SEQ ID NO: 11 contains the DAS-PZ-12709 SNP and flanking sequence.

SEQ ID NO: 12 contains the DAS-PZ-7838 SNP and flanking sequence.

SEQ ID NO: 13 contains the DAS-PZ-2135 SNP and flanking sequence.

SEQ ID NO: 14 contains the DAS-PZ-17603 SNP and flanking sequence.

SEQ ID NO: 15 contains the DAS-PZ-13243 SNP and flanking sequence.

SEQ ID NO: 16 contains the Mo17-13571 SNP and flanking sequence.

SEQ ID NO: 17 contains the KG-2626489 SNP and flanking sequence.

SEQ ID NO: 18 contains the DAS-PZ-44311 SNP and flanking sequence.

SEQ ID NO: 19 contains the Mo17-101779 SNP and flanking sequence.

SEQ ID NO: 20 contains the DAS-PZ-14396 SNP and flanking sequence.

SEQ ID NO: 21 contains the DAS-PZ-16143 SNP and flanking sequence.

SEQ ID NO: 22 contains the DAS-PZ-14379 SNP and flanking sequence.

SEQ ID NO: 23 contains the DAS-PZ-18284 SNP and flanking sequence.

SEQ ID NO: 24 contains the DAS-PZ-14406 SNP and flanking sequence.

SEQ ID NO: 25 contains the DAS-PZ-12076 SNP and flanking sequence.

SEQ ID NO: 26 contains the DAS-PZ-9937 SNP and flanking sequence.

SEQ ID NO: 27 contains the DAS-PZ-8939 SNP and flanking sequence.

SEQ ID NO: 28 contains the DAS-PZ-12401 SNP and flanking sequence.

SEQ ID NO: 29 contains the DAS-PZ-14269 SNP and flanking sequence.

SEQ ID NO: 30 contains the DAS-PZ-4624 SNP and flanking sequence.

SEQ ID NO: 31 contains the Mo17-138495NP and flanking sequence.

SEQ ID NO: 32 contains the DAS-PZ-4414 SNP and flanking sequence.

SEQ ID NO: 33 contains the DAS-PZ-4181 SNP and flanking sequence.

SEQ ID NO: 34 contains the Mo17-100863 SNP and flanking sequence.

SEQ ID NO: 35 contains the Mo17-102037 SNP and flanking sequence.

SEQ ID NO: 36 contains the DAS-PZ-40282 SNP and flanking sequence.

SEQ ID NO: 37 contains the DAS-PZ-915 SNP and flanking sequence.

SEQ ID NO: 38 contains the DAS-PZ-8563 SNP and flanking sequence.

SEQ ID NO: 39 contains the Mo17-13449 SNP and flanking sequence.

SEQ ID NO: 40 contains the DAS-PZ-2605 SNP and flanking sequence.

SEQ ID NO: 41 contains the DAS-PZ-19040 SNP and flanking sequence.

SEQ ID NO: 42 contains the Mo17-10473 SNP and flanking sequence.

SEQ ID NO: 43 contains the DAS-PZ-21 197 SNP and flanking sequence.

SEQ ID NO: 44 contains the Mo17-101840 SNP and flanking sequence.

SEQ ID NO: 45 contains the DAS-PZ-16358 SNP and flanking sequence.

SEQ ID NO: 46 contains the Mo17-11830 SNP and flanking sequence.

SEQ ID NO: 47 contains the Mo17-1107 SNP and flanking sequence.

SEQ ID NO: 48 contains the DAS-PZ-11065 SNP and flanking sequence.

SEQ ID NO: 49 contains the DAS-PZ-34879 SNP and flanking sequence.

SEQ ID NO: 50 contains the DAS-PZ-6897 SNP and flanking sequence.

SEQ ID NO: 51 contains the KG-2572840 SNP and flanking sequence.

SEQ ID NO: 52 contains the DAS-PZ-6618 SNP and flanking sequence.

SEQ ID NO: 53 contains the DAS-PZ-386 SNP and flanking sequence.

SEQ ID NO: 54 contains the Mo17-1333 SNP and flanking sequence.

SEQ ID NO: 55 contains the Mo17-14260 SNP and flanking sequence.

SEQ ID NO: 56 contains the Mo17-14261 SNP and flanking sequence.

SEQ ID NO: 57 contains the Mo17-12340 SNP and flanking sequence.

SEQ ID NO: 58 contains the DAS-PZ-1425 SNP and flanking sequence.

SEQ ID NO: 59 contains the DAS-PZ-14198 SNP and flanking sequence.

SEQ ID NO: 60 contains the Mo17-102555 SNP and flanking sequence.

SEQ ID NO: 61 through SEQ ID NO: 291 are the forward and reverse primers for the markers disclosed in Tables 2 and 3.

### DETAILED DESCRIPTION OF THE INVENTION

The present invention provides methods for identifying and selecting maize plants with increased culturability and transformability. The following definitions are provided as an aid to understand the invention.

The term “allele” refers to one of two or more different nucleotide sequences that occur at a specific locus.

An “amplicon” is amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid for a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

The term “assemble” applies to bacterial artificial clones (BACs) and their propensities for coming together to form contiguous stretches of DNA. A BAC “assembles” to a contig based on sequence alignment, if the BAC is sequenced, or via the alignment of its BAC fingerprint to the fingerprints of other BACs. The assemblies can be found using the Maize Genome Browser, which is publicly available on the internet.

An allele is “associated with” a trait when it is linked to it and when the presence of the allele is an indicator that the desired trait or trait form will occur in a plant comprising the allele.

The “B73 reference genome, version 2” is the physical and genetic framework of the maize B73 genome. It is the result of a sequencing effort utilizing a minimal tiling path of approximately 19,000 mapped BAC clones, and focusing on producing high-quality sequence coverage of all identifiable gene-containing regions of the maize genome. These regions were ordered, oriented, and along with all of the intergenic sequences, anchored to the extant physical and genetic maps of the maize genome. It can be accessed using a genome browser, the Maize Genome Browser, that is publicly available on the internet that facilitates user interaction with sequence and map data.

A “bacterial artificial chromosome (BAC)”is a cloning vector derived from the naturally occurring F factor of Escherichia coli. BACs can accept large inserts of DNA sequence. In maize, a number of BACs, or bacterial artificial chromosomes, each containing a large insert of maize genomic DNA, have been assembled into contigs (overlapping contiguous genetic fragments, or “contiguous DNA”).

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the Fl generation: the term “BC1” then refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

“Chromosomal interval” designates a contiguous linear span of genomic DNA that resides in a plant on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “complement” refers to a nucleotide sequence that is complementary to a given nucleotide sequence, i.e., the sequences are related by the base-pairing rules. The term “contiguous DNA” refers to overlapping contiguous genetic fragments. The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny. A “favorable allele” is the allele at a particular locus that confers, or contributes to, a desirable phenotype, e.g., increased culturability and transformability, or alternatively, is an allele that allows the identification of plants with decreased culturability and transformability that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or chromosomes) within a given species, generally depicted in a diagrammatic or tabular form. For each genetic map, distances between loci are measured by the recombination frequencies between them, and recombinations between loci can be detected using a variety of molecular genetic markers (also called molecular markers). A genetic map is a product of the mapping population, types of markers used, and the polymorphic potential of each marker between different populations. The order and genetic distances between loci can differ from one genetic map to another. However, information such as marker position and order can be correlated between maps by determining the physical location of the markers on the chromosome of interest, using the B73 reference genome, version 2, which is publicly available on the internet. One of ordinary skill in the art can use the publicly available genome browser to determine the physical location of markers on a chromosome.

The term “Genetic Marker” shall refer to any type of nucleic acid based marker, including but not limited to, Restriction Fragment Length Polymorphism (RFLP) (Botstein et al, 1998), Simple Sequence Repeat (SSR) (Jacob et al., 1991), Random Amplified Polymorphic DNA (RAPD) (Welsh et al., 1990), Cleaved Amplified Polymorphic Sequences (CAPS) (Rafalski and Tingey, 1993, Trends in Genetics 9:275-280), Amplified Fragment Length Polymorphism (AFLP) (Vos et al, 1995, Nucleic Acids Res. 23:4407-4414), Single Nucleotide Polymorphism (SNP) (Brookes, 1999, Gene 234:177-186), Sequence Characterized Amplified Region (SCAR) (Pecan and Michelmore, 1993, Theor. Appl. Genet, 85:985-993), Sequence Tagged Site (STS) (Onozaki et al. 2004, Euphytica 138:255-262), Single Stranded Conformation Polymorphism (SSCP) (Orita et al., 1989, Proc Natl Aced Sci USA 86:2766-2770). Inter-Simple Sequence Repeat (ISR) (Blair et al. 1999, Theor. Appl. Genet. 98:780-792), Inter-Retrotransposon Amplified Polymorphism (IRAP), Retrotransposon-Microsatellite Amplified Polymorphism (REMAP) (Kalendar et al., 1999, Theor. Appl. Genet 98:704-711), an RNA cleavage product (such as a Lynx tag), and the like.

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

A “heterotic group” comprises a set of genotypes that perform well when crossed with genotypes from a different heterotic group (Hallauer at al. (1998) Corn breeding, p. 463-564. In G. F. Sprague and J. W. Dudley (ed) Corn and corn improvement). Inbred lines are classified into heterotic groups, and are further subdivided into families within a heterotic group, based on several criteria such as pedigree, molecular marker-based associations, and performance in hybrid combinations (Smith at al. (1990) Theor. Appl. Gen. 80:833-840). The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the group i locus described herein may be introgressed into a recurrent parent that has reduced culturability and transformability. The recurrent parent line with the introgressed gene or locus then has increased culturability and transformability.

As used herein, the term “linkage” is used to describe the degree with which one marker locus is associated with another marker locus or some other locus (for example, a culturability or transformability locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits for both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same chromosome.) As used herein, linkage can be between two markers, or alternatively between a marker and a phenotype. A marker locus can be “associated with” (linked to) a trait, e.g., increased culturability and transformability. The degree of linkage of a molecular marker to a phenotypic trait is measured, e.g. as a statistical probability of co-segregation of that molecular marker with the phenotype.

Linkage disequilibrium is most commonly assessed using the measure r², which is calculated using the formula described by Hill, W. G. and Robertson, A, Theor Appl. Genet 38:226-231 (1988). When r²=1, complete LD exists between the two marker loci, meaning that the markers have not been separated by recombination and have the same allele frequency. Values for r² above 1/3 indicate sufficiently strong LD to be useful for mapping (Ardlie at al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

The “logarithm of odds (LOD) value” or “LOD score” (Risch, Science 255:803-804 (1992)) is used in interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. A “locus” is a position on a chromosome where a gene or marker is located. “Maize” refers to a plant of the Zea mays L. ssp. mays and is also known as “corn”. The term “maize plant” includes: whole maize plants, maize plant cells, maize plant protoplast, maize plant cell or maize tissue cultures from which maize plants can be regenerated, maize plant calli, and maize plant cells that are intact in maize plants or parts of maize plants, such as maize seeds, maize cobs, maize flowers, maize cotyledons, maize leaves, maize stems, maize buds, maize roots, maize root tips, and the like.

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, AFLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods. A large number of maize molecular markers are known in the art, and are published or available from various sources, such as the Maize GDB Internet resource and the Arizona Genomics Institute Internet resource run by the University of Arizona.

Markers corresponding to genetic polymorphisms between members of a population can be detected by methods well-established in the art. These include, e.g., DNA sequencing, PCR-based sequence specific amplification methods, detection of RFLPs, detection of isozyme markers, detection of polynucleotide polymorphisms by allele specific hybridization (ASH), detection of amplified variable sequences of the plant genome, detection of self-sustained sequence replication, detection of SSRs, detection of SNPs, or detection of AFLPs. Well established methods are also known for the detection of expressed sequence tags (ESTs) and SSR markers derived from EST sequences and RAPDs.

A “marker allele”, alternatively an “allele of a marker locus”, can refer to one of a plurality of polymorphic nucleotide sequences found at a marker locus in a population that is polymorphic for the marker locus.

“Marker assisted selection” (or MAS) is a process by which phenotypes are selected based on marker genotypes.“Marker assisted counter-selection” is a process by which marker genotypes are used to identify plants that will not be selected, allowing them to be removed from a breeding program or planting.

A “marker locus” is a specific chromosome location in the genome of a species when a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

A “marker probe” is a nucleic add sequence or molecule that can be used to identify the presence of a marker locus, e.g., a nucleic acid probe that is complementary to a marker locus sequence, through nucleic add hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e. genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a via a plant without the insertion. Thus, the marker need only indicate whether the indel region is present or absent. Any suitable marker detection technology may be used to identify such a hybridization marker, e.g., SNP technology is used in the examples provided herein.

“Nucleotide sequence”, “polynucleotide”, “nucleic acid sequence”, and “nucleic acid fragment” are used interchangeably and refer to a polymer of RNA or DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate. “G” for guanylate or deoxyguanylate. “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “phenotype”, or “phenotypic trait” or “trait” refers to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

“Plant tissue culture” refers to a collection of techniques used to maintain or grow plant cells, tissues or organs under sterile conditions on a nutrient culture medium of known composition. Plant tissue culture relies on the fact that many plant cells have the ability to regenerate a whole plant. Different techniques in plant tissue culture may offer certain advantages over traditional methods of propagation.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The “probability value” or “p-value” is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant”. In some embodiments, a probability score of 0.05 (p=0.05, or a 5% probability) of random assortment is considered a significant indication of co-segregation. However, an acceptable probability can be any probability of less than 50% (p=0.5). For example, a significant probability can be less than 0.25, less than 0.20, less than 0.15, less than 0.1, less than 0.05, less than 0.01, or less than 0.001.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

A “single nucleotide polymorphism (SNP)” is an allelic single nucleotide—A, T, C or G—variation within a DNA sequence representing one locus of at least two individuals of the same species,. For example, two sequenced DNA fragments representing the same locus from at least two individuals of the same species, AAGCCTA to AAGCTTA, contain a difference in a single nucleotide.

“Regeneration” is the process of growing a plant from a plant cell (e.g., plant protoplast, callus or explant).

The “Stiff Stalk” heterotic group represents a major heterotic group in the northern U.S. and Canadian corn growing regions. It can also be referred to as the Iowa Stiff Stalk Synthetic for BSSS heterotic group.

The term “transformation” refers to a process of introducing a DNA sequence or construct (e.g., a vector or expression cassette) into a cell or protoplast in which that exogenous DNA is incorporated into a chromosome or is capable of autonomous replication.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances.

Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50 of the probes are occupied at equilibrium), Stringent conditions will be those in which the salt concentration is less than about 1.0 M sodium ion, typically about 0.01 to 1.0 M sodium on concentration (or other salts) at pH 7.0 to 8.3, and the temperature is at least about 30° C. for short probes (e.g., 10 to 50 nucleotides) and at least about 60° C. for long probes (e.g. greater than 50 nucleotides). Stringent conditions may also be achieved with the addition of destabilizing agents such as form amide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SOS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10), Default parameters for pairwise alignments and calculation of percent identity of protein sequences using the Clustal V method are KTUPLE=1, GAP PENALTY=3, WINDOW=5 and DIAGONALS SAVED=5. For nucleic adds these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Before describing the present invention in detail, it should be understood that this invention is not limited to particular embodiments. It also should be understood that the terminology used herein is for the purpose of describing particular embodiments, and is not intended to be limiting. As used herein and in the appended claims, terms in the singular and the singular forms “a”, “an” and “the”, for example, include plural referents unless the content clearly dictates otherwise. Thus, for example, reference to “plant”, “the plant” or “a plant” also includes a plurality of plants. Depending on the context, use of the term “plant” can also include genetically similar or identical progeny of that plant. The use of the term “a nucleic acid” optionally includes many copies of that nucleic acid molecule.

### Genetic Mapping

It has been recognized for quite some time that specific genetic loci correlating with particular phenotypes, such as increased culturability and transformability, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as increased culturability and transformability. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference.

Two such methods used to detect trait loci of interest are: 1) Population-based association analysis and 2) Traditional linkage analysis. In a population-based association analysis, lines are obtained from pre-existing populations with multiple founders, e.g. elite breeding lines. Population-based association analyses rely on the decay of linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each, marker locus for each line in the subpopulation. A significant marker-trait association indicates the dose proximity between the marker locus and one or more genetic loci that are involved in the expression of that trait.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

As described above, methods well known in the art are available for detecting molecular markers or clusters of molecular markers that cosegregate with a trait of interest, such as increased culturability and transformability. However, certain experimental processes, such as the acts of culture, transformation and regeneration, apply a selection pressure on the genetically segregating population of plants so that only the plants containing the genetic regions for the trait of interest are able to survive and become BC₁ plants. Subsequently, if a genetic locus is important for the trait of interest, then an allele carried by one of the parents would be selected. Therefore, when the genome is scanned after the selection pressure is applied, alleles that are important for the trait of interest occur at a frequency of greater than 50%. Markers showing a significant deviation to greater than 50%, represent loci showing positive effects of selection, and can be identified using a Chi-square test (p<0.05). Such methods for molecular marker detection are described within.

### Markers Associated with Increased Culturability and Transformability

Markers associated with increased culturability and transformability are identified herein. The methods involve detecting the presence of at least four marker alleles associated with increased culturability and transformability in the germplasm of a maize plant. The marker loci can be selected from each of ten marker nucleic acid groups (i)-(x) provided in Table 2: (i) DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624; (ii) Mo17-13849, DAS-PZ-4414, DAS-PZ-4181, Mo17-100863, Mo17-102037, DAS-PZ-40282, DAS-PZ-915, DAS-PZ-8563, and PZA00991; (iii) Mo17-13449, DAS-PZ-2605, and DAS-PZ-19040;(iv) Mo17-10473, PZA00749, DAS-PZ-21197, Mo17-101840, DAS-PZ-16358, Mo17-11830, and PZA03070; (v) Mo17-1107, DAS-PZ-11065, magi73697, magi94661, and DAS-PZ-34879; (vi) PZA00089, PZA02462, DAS-PZ-6897, PZA02676, and KG-2572840; (vii) PZA01304 and magi95039; (viii) DAS-PZ-6618 and magi91369; (ix) PZA01297, DAS-PZ-386, Mo17-1333, PHM448, PZA03697, Mo17-14260, and Mo17-14261; and, (x) PZB01899, Mo17-12340, DAS-PZ-1425, DAS-PZ-14198, and Mo17-102555, and any other marker linked to these markers (linked markers can be determined from the Maize GDB resources).

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

Other markers linked to the markers listed in Table 2 can be used to predict culturability and transformability in a maize plant. This includes any marker within 50 cM of markers within groups (i)-(x): (i) DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624; (ii) Mo17-13849, DAS-PZ-4414, DAS-PZ-4181, Mo17-100863, Mo17-102037, DAS-PZ-40282, DAS-PZ-915, DAS-PZ-8563, and PZA00991; (iii) Mo17-13449, DAS-PZ-2605, and DAS-PZ-19040; (iv) Mo17-10473, PZA00749, DAS-PZ-21197, Mo17-101840, DAS-PZ-16358, Mo17-11830, and PZA03070; (v) Mo17-1107, DAS-PZ-11065, magi73697, magi94661, and DAS-PZ-34879 ; (vi) PZA00089, PZA02462, DAS-PZ-6897, PZA02676, and KG-2572840; (vii) PZA01304 and magi95039; (viii) DAS-PZ-6618 and magi91369; (ix) PZA01297, DAS-PZ-386, Mo17-1333, PHM448, PZA03697, Mo17-14260, and Mo17-14261; and, (x) PZB01899, Mo17-12340, DAS-PZ-1425, DAS-PZ-14198, and Mo17-102555, the markers associated with culturability and transformability. The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can show co-segregation with increased culturability and transformability, it is important to note that the marker locus is not necessarily responsible for the expression of the increased culturability and transformability phenotype. For example, it is not a requirement that the marker polynucleotide sequence be part of a gene that imparts increased culturability and transformability (for example, be part of the gene open reading frame). The association between a specific marker allele and the increased culturability and transformability phenotype is due to the original “coupling” linkage phase between the marker allele and the allele in the ancestral maize line from which the allele originated. Eventually, with repeated recombination, crossing over events between the marker and genetic locus can change this orientation. For this reason, the favorable marker allele may change depending on the linkage phase that exists within the donor parent used to create segregating populations. This does not change the fact that the marker can be used to monitor segregation of the phenotype. It only changes which marker allele is considered favorable in a given segregating population.

The present invention includes isolated nucleic acid molecules. Such molecules include those nucleic acid molecules capable of detecting a polymorphism genetically or physically linked to a culturability and transformability locus. Such molecules can be referred to as markers. Additional markers can be obtained that are linked to a culturability and transformability locus selected from the group i, ii, iii, iv, v, vi, vii, viii, ix, or x by available techniques. In one aspect, the nucleic acid molecule is capable of detecting the presence or absence of a marker located less than 30, 20, 10, 5, 2, or 1 cM from a culturability and transformability locus. In another aspect, the nucleic acid molecule is capable of detecting a marker in a locus selected from the group i, ii, iii, iv, v, vi, vii, viii, ix, or x. In a further aspect, a nucleic acid molecule is selected from the group consisting of SEQ ID NO: 1 through SEQ ID NO: 60, fragments thereof, complements thereof, and nucleic acid molecules capable of specifically hybridizing to one or more of these nucleic acid molecules. In another aspect, a nucleic acid molecule is selected from the publicly available markers listed in Table 2, fragments thereof, complements thereof, and nucleic acid molecules capable of specifically hybridizing to one or more of these nucleic acid molecules.

A marker of the invention can also be a combination of alleles at marker loci, otherwise known as a haplotype. The skilled artisan would expect that there might be additional polymorphic sites at marker loci in and around the culturability and transformability markers identified herein, wherein one, or more polymorphic sites is in linkage disequilibrium (LD) with an allele associated with increased culturability and transformability. Two particular alleles at different polymorphic sites are said to be in LD if the presence of the allele at one of the sites tends to predict the presence of the allele at the other site on the same chromosome (Stevens, Mol. Diag. 4:309-17 (1999)).

Marker Assisted Selection

Molecular markers can be used in a variety of, plant breeding applications (e.g. see Staub et al. (1996) Hortscience 729-741; Tanksley (1983) Plant Molecular Biology Reporter 1: 3-8). One of the main areas of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true with traits that are difficult to phenotype due to their dependence on environmental conditions. This category includes traits related to the resistance to biotic and abiotic stresses. This category also includes traits that are very expensive to phenotype because of laborious artificial inoculation or maintenance of managed stress environments. Another category of traits includes those which are associated with destruction of plant per se. Destructive phenotyping has been a bottleneck to implement MAS for the seed quality traits. Because DNA marker assays are not environmentally dependent, are robust, reliable, less laborious, less costly, and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will avow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with, markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of the B73 reference genome, version 2 and the integrated linkage maps of the maize genome containing increasing densities of public maize markers, has facilitated maize genetic mapping and MAS. See, e.g. the IBM2 Neighbors maps, which are available online on the Maize GDB website.

The key components to the implementation of MAS are (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 by or less (Tautz (1989) Nucleic Acid Research 17: 6463-6471; Wang et al. (1994) Theoretical and Applied Genetics, 88:1-6). Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396), SSRs are highly suited to mapping and MAS as they are multi-allelic, codominant, reproducible and amenable to high throughput automation (Rafalski et al. (1996) Generating and using DNA markers in plants. In Non-mammalian genomic analysis: a practical guide. Academic Press, pp 75-135).

Various types of SSR markers can be generated, and SSR profiles from resistant lines can be obtained by gel electrophoresis of the amplification products. Scoring of marker genotype is based on the size of the amplified fragment. An SSR service for maize is available to the public on a contractual basis by DNA Landmarks in Saint-Jean-sur-Richelieu, Quebec, Canada.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp, 475-492: Shi (2001) Clin Chem 47, pp. 164-172; Kwok (2000) Pharmacogenomics 1, pp. 95-100: Bhattramakki and Rafalski (2001) Discovery and application of single nucleotide polymorphism markers in plants. In: R, J Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), Invader® (Third Wave Technologies), SnapShot® (Applied Biosystems), Taqman® (Applied Biosystems) and Beadarrays™ (Illumina)

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than, single SNPs and can be more descriptive of any particular genotype. For example, single SNP may be allele ‘T’ for a specific line or variety with increased culturability and transformability, but the allele ‘T’ might also occur in the maize breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

The sequences listed in Table 2 can be readily used to obtain additional polymorphic SNPs (and other markers) linked to the markers listed in this disclosure that are associated with increased culturability and transformability. Markers associated with increased culturability and transformability can be hybridized to BACs or other genomic libraries, or electronically aligned with genome sequences, to find new sequences in the same approximate location as the described markers.

In addition to SSRs, RFLPs and SNPs, as described above, other types of molecular markers are also widely used, including but not limited to, markers derived from EST sequences, RAPDs, and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the maize species, or even across other species whose genomes share some level of colinearity at macro- and micro-level with maize, such as rice and sorghum.

In general, MAS uses polymorphic markers that have been identified as having a significant likelihood of co-segregation with increased culturability and transformability. Such markers are presumed to map near quantitative trait loci (QTL), give the plant its increased culturability and transformability phenotype, and are considered indicators, or markers, for the desired trait. Markers test maize plants for the presence of a desired allele, and those which contain a desired genotype at one or more loci are expected to transfer the desired genotype, along with a desired phenotype, to their progeny. The means to identify maize plants that have increased culturability and transformability by identifying plants that have a specified allele at from each of the ten marker nucleic acid groups (i)-(x) described herein, including: (i) DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624; (ii) Mo17-13849, DAS-PZ-4414, DAS-PZ-4181, Mo17-100863, Mo17-102037, DAS-PZ-40282, DAS-PZ-915, DAS-PZ-8563, and PZA00991; (iii) Mo17-13449, DAS-PZ-2605, and DAS-PZ-19040; (iv) Mo17-10473, PZA00749, DAS-PZ-21197, Mo17-101840, DAS-PZ-16358, Mo17-11830, and PZA03070; (v) Mo17-1107, DAS-PZ-11065, magi73697, magi94661, and DAS-PZ-34879 ; (vi) PZA00089, PZA02462, DAS-PZ-6897, PZA02676, and KG-2572840; (vii) PZA01304 and magi95039; (viii) DAS-PZ-6618 and magi91369; (ix) PZA01297, DAS-PZ-386, Mo17-1333, PHM448, PZA03697, Mo17-14260, and Mo17-14261; and, (x) PZB01899, Mo17-12340, DAS-PZ-1425, DAS-PZ-14198, and Mo17-102555.

The ten marker nucleic acid groups presented herein finds use in MAS to select plants that demonstrate increased culturability and transformability. Any marker that listed in Tables 2 and 3 can be used for this purpose. In addition, haplotypes comprising alleles at one or more marker loci within each of the ten marker nucleic acid groups (i)-(x) can be used to introduce a increased culturability and transformability trait into maize lines or varieties. Any allele or haplotype that is in linkage disequilibrium with an allele associated with increased culturability and transformability can be used in MAS to select plants with increased culturability and transformability.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

### Example 1

### Populations for Identification of SNP Markers Associated with Increased Culturability and Transformability

Initial crosses were made between the highly transformable line A188 and 2 Dow AgroSciences (DAS) elite inbred lines. The F₁ plants from these populations were then backcrossed to the elite lines, and immature BC₁ (Backcross 1) embryos were harvested, cultured, and regenerated into BC₁ plants. Leaf tissue from the regenerated plants was collected and DNA was extracted using the Biocel 1800 robotic platform (Agilent Technologies, Santa Clara, Calif.) and the Qiagen MagAttract protocol (Qiagen, Valencia, Calif.).

### Example 2

### Embryo Collection

Ears from the 2 populations were surface-sterilized by immersion in a 20% solution of sodium hypochlorite (5%) and two drops of Tween 20, for 20-30 minutes, followed by three rinses in sterile water. Immature zygotic embryos (1.0-2.0 mm) were aseptically dissected from each ear and randomly distributed into micro-centrifuge tubes for Agrobacterium infection. Embryos were pooled in the cases where multiple ears were available from the same line.

### Example 3

### Screening for Regeneration Ability

Embryos from the different lines and crosses were screened for their ability to regenerate in culture. A small number of embryos (5-30) from each of the ears used for transformation were plated on two types of media (ZM00002234 or ZM00001341, Table 1.5 or 1.4) lacking the selective agent. Cultures were incubated in the dark for 14 days at 28° C. Proliferated embryos were subcultured on the same type of media and incubated in the dark for another 14 days at 28° C. Embryogenic callus was transferred to regeneration media (ZM00002388, Table 1.8) and incubated under 16/8 hours (h) light/dark with light intensity of 80-100 micromoles per second per meter squared (μmol m⁻² s⁻¹) for 10-14 days at 28° C. Calli with shoots initiated were transferred to a second type of regeneration media (ZM00002242, Table 1.10) and incubated under 16/8 h light/dark with light intensity of 80-100 μmol m⁻² s⁻¹ for 10-14 days at 28° C. Regeneration frequency was estimated as the number of embryos that regenerated at least one shoot divided by the number of embryos plated.

### Example 4

### Transformation

### Example 4.1

### Agrobacterium strain and construct

Agrobacterium tumefaciens strain LBA4404 carrying the super binary vector pDAB 1405 was used for all the transformation experiments. The pDAB 1405 construct contains GFP gene v.2 under the control of ZmUbil promoter and its intron and PAT gene v.3 under the control of OsActin1 promoter and its intron. The two genes and promoters were flanked by RB7 MARs sequences (FIG. 1).

### Example 4.2

### Agrobacterium Culture Initiation

Glycerol stocks of the superbinary vector pDAB 1405 in the host Agrobacterium tumefaciens strain LBA4404 were obtained from the DAS Research Culture Collection. Streaked plates were made using AB minimal medium (AT00002172, Table 1.13) containing 100 mg/L spectinomycin , 250 mg/L streptomycin, and 10 mg/L tetracycline, and grown at 20° C. for 3-4 days. A single colony was then picked and streaked onto YEP plates (AT0002170, Table 1.14) containing the same antibiotics and incubated at 28° C. for 1-2 days.

### Example 4.3

### Agrobacterium Co-cultivation

On the experiment day, Agrobacterium colonies were taken from the YEP plate, suspended in 7-10 ml of infection medium (ZM00002231, Table 1.1) in a 50 ml tube, and the cell density was adjusted to OD₅₅₀=1.2-1.4 using a spectrophotometer. The Agrobacterium cultures were placed on a rotary shaker at 100 revolutions per minute (rpm) while embryo dissection was performed. Immature zygotic embryos between 1.5-2.0 mm in size were isolated from the sterilized maize kernels and placed in 1 ml of the infection medium (about 30-130 embryos in a 2.0 ml Eppendorf tube), followed by one wash with the same medium. The Agrobacterium suspension (1.0 ml) was added to each tube; the tubes were inverted for 20 times, and then allowed to sit for 5 minutes at room temperature. The embryos were then transferred onto co-cultivation media (ZM00002232 or ZM00001358, Table 1.2 or 1.3). The embryos were then oriented with the scutellum facing up using a microscope. After a 3 day co-cultivation at 20° C., transient expression of the green fluorescence protein (GFP) transgene was observed to validate Agrobacterium infection.

### Example 4.4

### Callus Selection and Regeneration of Transgenic Events

Following the co-cultivation period, embryos were transferred to resting media (ZM00002234 or ZM00001341, Table 1.5 or 1.4) containing the antibiotic cefotaxime, and incubated in the dark for 7 days at 28° C. Embryos were then transferred onto Selection 1 media (ZM00002240 or ZM00002180, Table 1.6 or 1.7) containing 3 mg/L Bialaphos as the selective agent for the introduced pat gene, and incubated in the dark for 14 days at 28° C. Proliferating embryogenic calli expressing GFP were cut under the stereomicroscope into smaller pieces (2-3 mm), transferred onto selection media containing 3 mg/L Bialaphos and incubated in the dark for another 10-14 days at 28° C. All the pieces cut from one embryo were circled with a black marker and considered as one event. This selection step allowed the callus to further proliferate and enhanced the uniformity of stable GFP expression. The callus selection period lasted for approximately four to six weeks. Proliferating, embryogenic calli expressing GFP were transferred onto Regeneration 1 media (ZM00002254, Table 1.9) containing 3 mg/L Bialaphos and cultured in the dark for 7-10 days at 28° C. This period was essential for the maturation of embryogenic callus.

Embryogenic calli with shoots initiated were transferred onto Regeneration 2 media (ZM00002242 or ZM00002255, Table 1.10 or 1.11) without Bialaphos. The cultures were incubated under 16/8 h light/dark with light intensity of 80-100 μmol m⁻² s⁻¹ for 10-14 days at 28° C. Small shoots with primary roots were then transferred to shoot elongation and rooting media (ZM00002238, Table 1.12) in Magenta boxes and incubated under 16/8 h light/dark for 7-10 days at 28° C. Putative plantlets were confirmed for GFP expression and then scored as transgenic events.

TABLE 1
Media formulations

| | | | | |
|---|---|---|---|---|
| ID | pH | Ingredient | Conc. | Units |

1.1. Infection

| | | | | |
|---|---|---|---|---|
| ZM00002231 | 5.2 | MS BASAL SALTS | 4.33 | g/L |
| | | CHU N6 VITAMIN SOLUTION | 1 | mL/L |
| | | (1000X) | | |
| | | SUCROSE | 68.5 | g/L |
| | | D(+)GLUCOSE | 36 | g/L |
| | | 2,4-D 10 MG/ML STOCK | 150 | μL/L |

1.2. Co-cultivation

| | | | | |
|---|---|---|---|---|
| ZM00002232 | 5.8 | MS BASAL SALTS | 4.33 | g/L |
| | | 2,4-D 10 MG/ML STOCK | 200 | μL/L |
| | | L-PROLINE | 700 | mg/L |
| | | MES | 500 | mg/L |
| | | CHU N6 VITAMIN SOLUTION | 1 | mL/L |
| | | (1000X) | | |
| | | SUCROSE | 20 | g/L |
| | | MYO-INOSITOL | 100 | mg/L |
| | | GLUCOSE | 10 | g/L |
| | | AGAR | 7 | g/L |
| | | ACETOSYRINGONE 200 MM | 200 | μM |

1.3. Co-cultivation

| | | | | |
|---|---|---|---|---|
| ZM00001358 | 5.8 | MS BASAL SALT | 4.33 | g/L |
| | | L-PROLINE | 700 | mg/L |
| | | MYO-INOSITOL | 100 | mg/L |
| | | CASEIN ENZYMATIC | 100 | mg/L |
| | | HYDROLYSATE | | |
| | | SUCROSE | 30 | g/L |
| | | DICAMBA 50 MG/ML | 3.3 | mg/L |
| | | GELRITE 714246 | 2.3 | g/L |
| | | SILVER NITRATE | 15 | mg/L |
| | | ISU MODIFIED MS VITAMIN | 1 | mg/L |
| | | (1000X) | | |
| | | ACETOSYRINGONE 100 MM | 100 | μM |
| | | L-CYSTEINE | 300 | mg/L |

1.4. Resting

| | | | | |
|---|---|---|---|---|
| ZM00001341 | 5.8 | MS BASAL SALT | 4.33 | g/L |
| | | L-PROLINE | 700 | mg/L |
| | | MES | 500 | mg/L |
| | | MYO-INOSITOL | 100 | mg/L |
| | | CASEIN ENZYMATIC | 100 | mg/L |
| | | HYDROLYSATE | | |
| | | SUCROSE | 30 | g/L |
| | | DICAMBA 50 MG/ML | 3.3 | mg/L |
| | | GELRITE 714246 | 2.3 | g/L |
| | | SILVER NITRATE | 15 | mg/L |
| | | ISU MODIFIED MS VITAMIN | 1 | mg/L |
| | | (1000X) | | |
| | | CEFOTAXIME 250 MG/ML | 250 | mg/L |

1.5. Resting

| | | | | |
|---|---|---|---|---|
| ZM00002234 | 5.8 | MS BASAL SALTS | 4.33 | g/L |
| | | 2,4-D 10 MG/ML STOCK | 150 | μL/L |
| | | L-PROLINE | 700 | mg/L |
| | | MES | 500 | mg/L |
| | | CHU N6 VITAMIN SOLUTION | | |
| | | (1000X) | 1 | mL/L |
| | | SUCROSE | 30 | g/L |
| | | MYO-INOSITOL | 100 | mg/L |
| | | AGAR | 7 | g/L |
| | | CARBENICILLIN 250 MG/ML | 200 | mg/L |
| | | SILVER NITRATE | 0.85 | mg/L |

1.6. 3 mg/L Bialaphos selection medium

| | | | |
|---|---|---|---|
| ZM00002240 | MS BASAL SALTS | 4.33 | g/L |
| | 2,4-D 10 MG/ML STOCK | 150 | μL/L |
| | L-PROLINE | 700 | mg/L |
| | MES | 500 | mg/L |
| | CHU N6 VITAMIN SOLUTION | 1 | mL/L |
| | (1000X) | | |
| | SUCROSE | 30 | g/L |
| | MYO-INOSITOL | 100 | mg/L |
| | AGAR | 7 | g/L |
| | CARBENICILLIN 250 MG/ML | 200 | mg/L |
| | SILVER NITRATE | 0.85 | mg/L |
| | BIALAPHOS 5.0 MG/ML | 1.5 | mg/L |

1.7. 3 mg/L Bialaphos selection medium

| | | | | |
|---|---|---|---|---|
| ZM00002180 | 5.8 | MS BASAL SALT | 4.33 | g/L |
| | | L-PROLINE | 700 | mg/L |
| | | MES | 500 | mg/L |
| | | MYO-INOSITOL | 100 | mg/L |
| | | CASEIN ENZYMATIC | 100 | mg/L |
| | | HYDROLYSATE | | |
| | | SUCROSE | 30 | g/L |
| | | DICAMBA 1 MG/ML STOCK | 3.3 | mL/L |
| | | GELRITE 714246 | 3 | g/L |
| | | SILVER NITRATE | 15 | mg/L |
| | | ISU MODIFIED MS VITAMIN | 1 | mg/L |
| | | (1000X) | | |
| | | CEFOTAXIME 250 MG/ML | 250 | mg/L |
| | | BIALAPHOS 5.0 MG/ML | 3 | mg/L |

1.8. Regeneration 1 KD

| | | | | |
|---|---|---|---|---|
| ZM00002388 | 5.8 | MS BASAL SALTS WITH | 4.33 | g/L |
| | | VITAMINS | | |
| | | MES | 500 | mg/L |
| | | L-PROLINE | 500 | mg/L |
| | | SUCROSE | 30 | g/L |
| | | AGAR | 6.5 | g/L |
| | | 2,4-D 1 MG/ML STOCK | 0.25 | mL/L |
| | | KINETIN 1 MG/ML STOCK | 0.5 | mL/L |
| | | CEFOTAXIME 250 MG/ML | 150 | mg/L |

1.9. Regeneration 1 + 3 mg/L Bialaphos

| | | | | |
|---|---|---|---|---|
| ZM00002254 | 5.8 | MS BASAL SALT | 4.33 | g/L |
| | | ISU MODIFIED MS VITAMIN | 1 | mg/L |
| | | (1000X) | | |
| | | SUCROSE | 60 | g/L |
| | | MYO-INOSITOL | 100 | mg/L |
| | | GELRITE 714246 | 2.5 | g/L |
| | | BIALAPHOS 5.0 MG/ML | 3 | mg/L |
| | | CEFOTAXIME 250 MG/ML | 250 | mg/L |

1.10. Regeneration 2

| | | | | |
|---|---|---|---|---|
| ZM00002242 | 5.8 | MS BASAL SALTS WITH | 4.43 | g/L |
| | | VITAMINS | | |
| | | MES | 500 | mg/L |
| | | SUCROSE | 30 | g/L |
| | | AGAR | 7 | g/L |

1.11. Regeneration 2

| | | | |
|---|---|---|---|
| ZM00002255 | MS BASAL SALT | 4.33 | g/L |
| | ISU MODIFIED MS VITAMIN | 1 | mg/L |
| | (1000X) | | |
| | SUCROSE | 30 | g/L |
| | MYO-INOSITOL | 100 | mg/L |
| | GELRITE 714246 | 2.5 | g/L |
| | CEFOTAXIME 250 MG/ML | 250 | mg/L |

1.12. Shoot elongation

| | | | | |
|---|---|---|---|---|
| ZM00002238 | 5.8 | MS BASAL SALTS WITH | 2.2 | g/L |
| | | VITAMINS | | |
| | | SUCROSE | 20 | g/L |
| | | AGAR | 6 | g/L |

1.13. AB + Antibiotics

| | | | | |
|---|---|---|---|---|
| AT00002172 | 7 | GLUCOSE | 5 | g/L |
| | | BACTO AGAR | 15 | g/L |
| | | AB MINIMAL BUFFER-MICRO | 1000 | mg/L |
| | | AB MINIMAL SALTS-MICRO | 1000 | mg/L |
| | | SPECTINOMYCIN 100 MG/ML | 100 | mg/L |
| | | STREPTOMYCIN 100 MG/ML | 250 | mg/L |
| | | TETRACYCLINE 10 MG/ML | 10 | mg/L |

1.14. YEP + Antibiotics

| | | | | |
|---|---|---|---|---|
| AT00002170 | 7 | BACTO-PEPTONE | 10 | g/L |
| | | YEAST EXTRACT | 10 | g/L |
| | | SODIUM CHLORIDE | 5 | g/L |
| | | BACTO AGAR | 15 | g/L |
| | | STREPTOMYCIN 250 MG/ML | 250 | mg/L |
| | | AI-MICRO | | |
| | | TETRACYCLINE 10 MG/ML | 10 | mg/L |
| | | SPECTINOMYCIN 100 MG/ML | 100 | mg/L |
| | | AI-MICRO | | |

### Example 5

### Genotyping the BC₁ Plants

For the first population, 173 BC₁ plants were regenerated from independent cultures, while 117 BC₁ plants were regenerated for the second population. The KBioscience Competitive Allele Specific PCR system, or KASPar™ (KBiosciences, Hertfordshire, UK), was used to genotype DNA from leaf tissue from the regenerated plants using 256 and 235 polymorphic markers for the first and second populations, respectively. The KASPar™ system is comprised of two components (1) the SNP specific assay (a combination of three unlabelled primers), and (2) the universal Reaction Mix, which contains all other required components including the universal fluorescent reporting system and a specially developed Taq polymerase. The three primers, allele-specific 1 (A1), allele-specific 2 (A2), and common (C1), or reverse, were designed using the assay design algorithm of the workflow manager, Kraken (KBiosciences, Hertfordshire, UK).

An Assay Mix of the 3 primers was made, consisting of 12 μM each of A1 and A2 and 30 μM of C1. The universal 1536 Reaction Mix was diluted to 1×. A volume of 2.0 μl of DNA diluted 1:20 from MagAttract extracted DNA was dispensed into PCR plates using a liquid handling robot and dried for 2 hours at 65° C. Next, 1.3 μl of 1×KASP 1536 Reaction Mix was added to the PCR plates. Plates were sealed using a Fusion heat sealer (Kbioscience, Hertfordshire, UK). Thermal cycling was completed in the Hydrocycler water bath thermal cycler (Kbioscience, Hertfordshire, UK) with the following conditions: initial denaturation and hot-start enzyme activation at 94° C. for 15 minutes followed by 10 cycles of denaturation at 94° C. for 20 seconds and touchdown over 65-57° C. for 60 seconds (dropping 0.8° C. per cycle). This was followed by 29 cycles of denaturation at 94° C. for 20 seconds and 57° C. annealing for 60 seconds.

KASPar™ uses the fluorophores FAM and VIC for distinguishing genotypes. The passive reference dye ROX is also used to allow normalization of variations in signal caused by differences in well-to-well liquid volume. In Kraken, the FAM and VIC data are plotted on the x- and y-axes, respectively. Genotypes can then be determined according to sample clusters.

### Example 6

### Statistical Analysis

The acts of culture, transformation and regeneration applied a selection pressure on the genetically segregating population of embryos so that only embryos containing the genetic regions important for tissue culture, transformation and regeneration were able to survive and become BC₁ plants. Subsequently, if a genetic locus is important for culture, transformation or regeneration, then an allele carried by one of the parents would be selected. Therefore, when the genome is scanned after culture and plant regeneration, alleles that are important for culturability and transformability occur at a frequency of greater than 50%. Markers showing a significant deviation to greater than 50%, represent loci showing positive effects of selection, and were identified using a Chi-square test (p<0.05). Chi-square analysis results identified ten marker nucleic acid groups (i)-(x) associated with increased culturability and transformability (Table 2).

TABLE 2
Summary of SNP markers associated with increased
culturability and transformability.

| Marker | Group | SEQ ID NO | SNP | Donor Allele |
|---|---|---|---|---|
| DAS-PZ-12690 | i | 1 | A/G | G |
| PZA03520 | i | * | A/G | A |
| DAS-PZ-9388 | i | 2 | A/G | A |
| DAS-PZ-17750 | i | 3 | T/G | T |
| DAS-PZ-20486 | i | 4 | C/G | C |
| DAS-PZ-10624 | i | 5 | A/G | G |
| DAS-PZ-16137 | i | 6 | A/T | A |
| Mo17-10690 | i | 7 | A/G | A |
| DAS-PZ-5621 | i | 8 | A/G | A |
| KG-2507169 | i | 9 | A/G | A |
| Mo17-1018 | i | 10 | A/C | C |
| DAS-PZ-12709 | i | 11 | A/T | T |
| DAS-PZ-7838 | i | 12 | A/T | A |
| DAS-PZ-2135 | i | 13 | T/G | G |
| DAS-PZ-17603 | i | 14 | A/G | G |
| DAS-PZ-13243 | i | 15 | A/G | G |
| Mo17-13571 | i | 16 | T/C | T |
| KG-2626489 | i | 17 | A/C | C |
| magi84607 | i | * | T/C | T |
| DAS-PZ-44311 | i | 18 | T/C | T |
| Mo17-101779 | i | 19 | T/G | T |
| DAS-PZ-14396 | i | 20 | A/G | A |
| DAS-PZ-16143 | i | 21 | C/G | G |
| DAS-PZ-14379 | i | 22 | A/G | G |
| DAS-PZ-18284 | i | 23 | C/G | G |
| DAS-PZ-14406 | i | 24 | A/C | C |
| DAS-PZ-12076 | i | 25 | T/G | G |
| DAS-PZ-9937 | i | 26 | A/C | C |
| DAS-PZ-8939 | i | 27 | T/C | CT |
| DAS-PZ-12401 | i | 28 | A/C | C |
| DAS-PZ-14269 | i | 29 | A/G | G |
| DAS-PZ-4624 | i | 30 | T/C | T |
| Mo17-13849 | ii | 31 | C/G | C |
| DAS-PZ-4414 | ii | 32 | A/T | T |
| DAS-PZ-4181 | ii | 33 | T/C | C |
| Mo17-100863 | ii | 34 | T/C | C |
| Mo17-102037 | ii | 35 | A/G | G |
| DAS-PZ-40282 | ii | 36 | T/C | T |
| DAS-PZ-915 | ii | 37 | C/G | G |
| DAS-PZ-8563 | ii | 38 | A/G | A |
| PZA00991 | ii | * | A/C | A |
| Mo17-13449 | iii | 39 | C/G | C |
| DAS-PZ-2605 | iii | 40 | C/G | C |
| DAS-PZ-19040 | iii | 41 | A/G | G |
| Mo17-10473 | iv | 42 | C/G | C |
| PZA00749 | iv | * | C/G | C |
| DAS-PZ-21197 | iv | 43 | T/C | C |
| Mo17-101840 | iv | 44 | T/G | T |
| DAS-PZ-16358 | iv | 45 | T/C | C |
| Mo17-11830 | iv | 46 | C/G | G |
| PZA03070 | iv | * | T/G | G |
| Mo17-1107 | v | 47 | C/G | G |
| DAS-PZ-11065 | v | 48 | T/G | T |
| magi73697 | v | * | T/G | G |
| magi94661 | v | * | A/G | G |
| DAS-PZ-34879 | v | 49 | C/G | G |
| PZA00089 | vi | * | A/C | A |
| PZA02462 | vi | * | A/C | C |
| DAS-PZ-6897 | vi | 50 | A/C | C |
| PZA02676 | vi | * | T/C | T |
| KG-2572840 | vi | 51 | A/G | G |
| PZA01304 | vii | * | A/G | A |
| magi95039 | vii | * | T/G | T |
| DAS-PZ-6618 | viii | 52 | A/G | G |
| magi91369 | viii | * | A/C | C |
| PZA01297 | ix | * | T/G | T |
| DAS-PZ-386 | ix | 53 | T/C | C |
| Mo17-1333 | ix | 54 | A/G | A |
| PHM448 | ix | * | A/G | A |
| PZA03697 | ix | * | A/C | A |
| Mo17-14260 | ix | 55 | T/G | T |
| Mo17-14261 | ix | 56 | T/C | T |
| PZB01899 | x | * | A/G | A |
| Mo17-12340 | x | 57 | A/G | A |
| DAS-PZ-1425 | x | 58 | C/G | G |
| DAS-PZ-14198 | x | 59 | T/C | T |
| Mo17-102555 | x | 60 | C/G | G |

*Sequence is available in public databases such as Maize GDB or Panzea.

### Example 7

### Marker Framework and Use for MAS

A set of common markers can be used to establish a framework for identifying markers linked to a QTL. Closely linked markers flanking the locus of interest that have alleles in linkage disequilibrium with a favorable allele at that locus may be effectively used to select for progeny plants with increased culturability and transformability. Thus, the markers described in herein, such as those listed in Table 2, as well as other markers genetically or physically mapped to the same chromosomal segment, may be used to select for maize plants with increased culturability and transformability. Typically, a set of these markers will be used (e.g. 2 or more, 3 or more, 4 or more, 5 or more) in the regions flanking the locus of interest. Optionally, a marker within the actual gene and/or locus may be used. Exemplary primers for amplifying and detecting genomic regions associated with increased culturability and transformability are given in Table 3.

Exemplary assays for detecting increased culturability and
transformability.

| | | SEQ ID NO | SEQ ID NO | SEQ ID NO |
|---|---|---|---|---|
| | | Allele | Allele | Common |
| | SEQ ID | specific | specific | Primer |
| Marker | NO | primer (A1) | primer (A2) | (C1) |
| DAS-PZ-12690 | 1 | 61 | 62 | 63 |
| PZA03520 | * | 64 | 65 | 66 |
| DAS-PZ-9388 | 2 | 67 | 68 | 69 |
| DAS-PZ-17750 | 3 | 70 | 71 | 72 |
| DAS-PZ-20486 | 4 | 73 | 74 | 75 |
| DAS-PZ-10624 | 5 | 76 | 77 | 78 |
| DAS-PZ-16137 | 6 | 79 | 80 | 81 |
| Mo17-10690 | 7 | 82 | 83 | 84 |
| DAS-PZ-5621 | 8 | 85 | 86 | 87 |
| KG-2507169 | 9 | 88 | 89 | 90 |
| Mo17-1018 | 10 | 91 | 92 | 93 |
| DAS-PZ-12709 | 11 | 94 | 95 | 96 |
| DAS-PZ-7838 | 12 | 97 | 98 | 99 |
| DAS-PZ-2135 | 13 | 100 | 101 | 102 |
| DAS-PZ-17603 | 14 | 103 | 104 | 105 |
| DAS-PZ-13243 | 15 | 106 | 107 | 108 |
| Mo17-13571 | 16 | 109 | 110 | 111 |
| KG-2626489 | 17 | 112 | 113 | 114 |
| magi84607 | * | 115 | 116 | 117 |
| DAS-PZ-44311 | 18 | 118 | 119 | 120 |
| Mo17-101779 | 19 | 121 | 122 | 123 |
| DAS-PZ-14396 | 20 | 124 | 125 | 126 |
| DAS-PZ-16143 | 21 | 127 | 128 | 129 |
| DAS-PZ-14379 | 22 | 130 | 131 | 132 |
| DAS-PZ-18284 | 23 | 133 | 134 | 135 |
| DAS-PZ-14406 | 24 | 136 | 137 | 138 |
| DAS-PZ-12076 | 25 | 139 | 140 | 141 |
| DAS-PZ-9937 | 26 | 142 | 143 | 144 |
| DAS-PZ-8939 | 27 | 145 | 146 | 147 |
| DAS-PZ-12401 | 28 | 148 | 149 | 150 |
| DAS-PZ-14269 | 29 | 151 | 152 | 153 |
| DAS-PZ-4624 | 30 | 154 | 155 | 156 |
| Mo17-13849 | 31 | 157 | 158 | 159 |
| DAS-PZ-4414 | 32 | 160 | 161 | 162 |
| DAS-PZ-4181 | 33 | 163 | 164 | 165 |
| Mo17-100863 | 34 | 166 | 167 | 168 |
| Mo17-102037 | 35 | 169 | 170 | 171 |
| DAS-PZ-40282 | 36 | 172 | 173 | 174 |
| DAS-PZ-915 | 37 | 175 | 176 | 177 |
| DAS-PZ-8563 | 38 | 178 | 179 | 180 |
| PZA00991 | * | 181 | 182 | 183 |
| Mo17-13449 | 39 | 184 | 185 | 186 |
| DAS-PZ-2605 | 40 | 187 | 188 | 189 |
| DAS-PZ-19040 | 41 | 190 | 191 | 192 |
| Mo17-10473 | 42 | 193 | 194 | 195 |
| PZA00749 | * | 196 | 197 | 198 |
| DAS-PZ-21197 | 43 | 199 | 200 | 201 |
| Mo17-101840 | 44 | 202 | 203 | 204 |
| DAS-PZ-16358 | 45 | 205 | 206 | 207 |
| Mo17-11830 | 46 | 208 | 209 | 210 |
| PZA03070 | * | 211 | 212 | 213 |
| Mo17-1107 | 47 | 214 | 215 | 216 |
| DAS-PZ-11065 | 48 | 217 | 218 | 219 |
| magi73697 | * | 220 | 221 | 222 |
| magi94661 | * | 223 | 224 | 225 |
| DAS-PZ-34879 | 49 | 226 | 227 | 228 |
| PZA00089 | * | 229 | 230 | 231 |
| PZA02462 | * | 232 | 233 | 234 |
| DAS-PZ-6897 | 50 | 235 | 236 | 237 |
| PZA02676 | * | 238 | 239 | 240 |
| KG-2572840 | 51 | 241 | 242 | 243 |
| PZA01304 | * | 244 | 245 | 246 |
| magi95039 | * | 247 | 248 | 249 |
| DAS-PZ-6618 | 52 | 250 | 251 | 252 |
| magi91369 | * | 253 | 254 | 255 |
| PZA01297 | * | 256 | 257 | 258 |
| DAS-PZ-386 | 53 | 259 | 260 | 261 |
| Mo17-1333 | 54 | 262 | 263 | 264 |
| PHM448 | * | 265 | 266 | 267 |
| PZA03697 | * | 268 | 269 | 270 |
| Mo17-14260 | 55 | 271 | 272 | 273 |
| Mo17-14261 | 56 | 274 | 275 | 276 |
| PZB01899 | * | 277 | 278 | 279 |
| Mo17-12340 | 57 | 280 | 281 | 282 |
| DAS-PZ-1425 | 58 | 283 | 284 | 285 |
| DAS-PZ-14198 | 59 | 286 | 287 | 288 |
| Mo17-102555 | 60 | 289 | 290 | 291 |

*Sequence is available in public databases such as Maize GDB or Panzea.

### Example 8

### Introgression of Increased Culturability and Transformability into a Corn Plant

Corn breeders can use the SNP markers provided in the present invention to introgress increased culturability and transformability traits into a corn plant. The markers provided in Table 3 can be used to monitor the introgression of culturability and transformability QTL into a corn plant.

The introgression of one or more culturability and transformability loci is achieved via one or more cycles of backcrossing to a recurrent parent with one or more preferred agronomic characteristics, accompanied by selection to retain the one or more culturability and transformability loci from the donor parent using the markers of the present invention. Introgression can be monitored by genotyping one or more plants and determining the allelic state of the one or more culturability and transformability loci. This backcross procedure is implemented at any stage in variety development and occurs in conjunction with breeding for one or more traits of interest including transgenic and nontransgenic traits.

Alternatively, a forward breeding approach is employed wherein one or more culturability and transformability loci can be monitored for successful introgression following a cross with a susceptible parent with subsequent generations genotyped for one or more culturability and transformability loci and for one or more additional traits of interest, including transgenic and nontransgenic traits.

We claim:

1. A method for selecting a plant having an altered culturability and transformability characteristic, the method comprising the steps of: a) detecting at least one marker nucleic acid; and, b) selecting a plant comprising the marker nucleic acid, thereby selecting a plant having the altered culturability and transformability characteristic.

2. The method of claim 1, wherein the plant is a maize plant.

3. The method of claim 2, wherein the altered culturability and transformability characteristic is increased culturability and transformability.

4. The method of claim 3, wherein the marker nucleic acid is selected from the group consisting of DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, and DAS-PZ-4624.

5. The method of claim 3, wherein the marker nucleic acid is selected from the group consisting of Mo17-13849, DAS-PZ-4414, DAS-PZ-4181, Mo17-100863, Mo17-102037, DAS-PZ-40282, DAS-PZ-915, DAS-PZ-8563, and PZA00991.

6. The method of claim 3, wherein the marker nucleic acid is selected from the group consisting of Mo17-13449, DAS-PZ-2605, and DAS-PZ-19040.

7. The method of claim 3, wherein the marker nucleic acid is selected from the group consisting of Mo17-10473, PZA00749, DAS-PZ-21197, Mo17-101840, DAS-PZ-16358, Mo17-11830, and PZA03070.

8. The method of claim 3, wherein the marker nucleic acid is selected from the group consisting of Mo17-1107, DAS-PZ-11065, magi73697, magi94661, and DAS-PZ-34879.

9. The method of claim 3, wherein the marker nucleic acid is selected from the group consisting of PZA00089, PZA02462, DAS-PZ-6897, PZA02676, and KG-2572840.

10. The method of claim 3, wherein the marker nucleic acid is selected from the group consisting of PZA01304 and magi95039.

11. The method of claim 3, wherein the marker nucleic acid is selected from the group consisting of DAS-PZ-6618 and magi91369.

12. The method of claim 3, wherein the marker nucleic acid is selected from the group consisting of PZA01297, DAS-PZ-386, Mo17-1333, PHM448, PZA03697, Mo17-14260, and Mo17-14261.

13. The method of claim 3, wherein the marker nucleic acid is selected from the group consisting of PZB01899, Mo17-12340, DAS-PZ-1425, DAS-PZ-14198, and Mo17-102555.

14. The method of claim 1, wherein at least two marker nucleic acids are selected.

15. The method of claim 1, wherein at least three marker nucleic acids are selected.

16. The method of claim 1, wherein at least four marker nucleic acids are selected.

17. The method of claim 1, wherein at least five marker nucleic acids are selected.

18. The method of claim 1, wherein at least six marker nucleic acids are selected.

19. The method of claim 1, wherein at least seven marker nucleic acids are selected.

20. The method of claim 1, wherein at least eight marker nucleic acids are selected.

21. The method of claim 1, wherein at least nine marker nucleic acids are selected.

22. The method of claim 1, wherein at least ten marker nucleic acids are selected.

23. A maize plant obtained by the method of claim 1.

24. A method for selecting a maize plant having increased culturability and transformability, the method comprising: a) detecting at least ten marker nucleic acids, wherein at least one marker nucleic acid is selected from each of ten marker nucleic acid groups (i)-(x):
(i) DAS-PZ-12690, PZA03520, DAS-PZ-9388, DAS-PZ-17750, DAS-PZ-20486, DAS-PZ-10624, DAS-PZ-16137, Mo17-10690, DAS-PZ-5621, KG-2507169, Mo17-1018, DAS-PZ-12709, DAS-PZ-7838, DAS-PZ-2135, DAS-PZ-17603, DAS-PZ-13243, Mo17-13571, KG-2626489, magi84607, DAS-PZ-44311, Mo17-101779, DAS-PZ-14396, DAS-PZ-16143, DAS-PZ-14379, DAS-PZ-18284, DAS-PZ-14406, DAS-PZ-12076, DAS-PZ-9937, DAS-PZ-8939, DAS-PZ-12401, DAS-PZ-14269, DAS-PZ-4624; and,
(ii) Mo17-13849, DAS-PZ-4414, DAS-PZ-4181, Mo17-100863, Mo17-102037, DAS-PZ-40282, DAS-PZ-915, DAS-PZ-8563, PZA00991; and,
(iii) Mo17-13449, DAS-PZ-2605, DAS-PZ-19040; and,
(iv) Mo17-10473, PZA00749, DAS-PZ-21197, Mo17-101840, DAS-PZ-16358, Mo17-11830, PZA03070; and,
(v) Mo17-1107, DAS-PZ-11065, magi73697, magi94661, DAS-PZ-34879; and,
(vi) PZA00089, PZA02462, DAS-PZ-6897, PZA02676, KG-2572840; and,
(vii) PZA01304, magi95039; and,
(viii) DAS-PZ-6618, magi91369; and,
(ix) PZA01297, DAS-PZ-386, Mo17-1333, PHM448, PZA03697, Mo17-14260, Mo17-14261; and,
(x) PZB01899, Mo17-12340, DAS-PZ-1425, DAS-PZ-14198, and Mo17-102555; and,
b) selecting a plant comprising the ten marker nucleic acids, thereby selecting a maize plant having increased culturability and transformability.

25. A maize plant obtained by the method of claim 24.
